(19) United States
(12) Patent Application Publication
(10) Pub. No.: US 2026/0250326 A1
(43) Pub. Date: Aug. 27, 2026

(54) MULTI-PATCH VACCINES AND IMMUNOGENIC COMPOSITIONS, METHODS OF PRODUCTION AND USES THEREOF

(71) Applicants: Sukrit SRIVASTAVA, Rae Bareli (IN); Helmholtz-Zentrum für Infektionsforschung GmbH, Braunschweig (DE)

(72) Inventors: Sukrit SRIVASTAVA, Rae Bareli (IN); Michael KOLBE, Rellingen (DE)

(21) Appl. No.: 18/023,576

(22) PCT Filed: Sep. 1, 2021
(86) PCT No.: PCT/IN2021/050841

§ 371 Date: Feb. 27, 2023

(30) Foreign Application Priority Data
Sep. 1, 2020 (IN) 202011037585
Sep. 3, 2020 (IN) 202011037939

(51) Int. Cl. C07K 14/005; A61K 39/00; A61K 39/215; G16B 30/10

(52) U.S. Cl. CPC C07K 14/005; A61K 39/215; G16B 30/10; A61K 2039/6031; C12N 2770/20022; C12N 2770/20034

(57) ABSTRACT

The present invention relates to a method for producing immunogenic compositions, such as vaccines, by identifying amino acid sequence of antigenic patches (Ag-Patches) from at least one proteome of a pathogen, derived from overlapping epitope clusters. In particular, the invention relates to the design of Multi-Patch Vaccines against viral, such as SARS-CoV-2, bacterial, fungi, parasite, pathogenic and non-pathogenic targets.

The present invention relates to a method for producing immunogenic compositions, such as vaccines, by identifying amino acid sequence of antigenic patches (Ag-Patches) from at least one proteome of a pathogen, derived from overlapping epitope clusters. In particular, the invention relates to the design of Multi-Patch Vaccines against viral, such as SARS-CoV-2, bacterial, fungi, parasite, pathogenic and non-pathogenic targets.

### BACKGROUND OF THE INVENTION

There are several strategies to develop vaccines, but majority of them are based on single proteins that happen to be mostly surface proteins of pathogens, since the proteins of pathogens that form interaction complexes with human cell proteins are considered most important for vaccine development. For example, in case of SARS-CoV-2 infection, the interaction of the spike(S) protein of SARS-CoV-2 and ACE2 on the host human cell surface leads to the initiation of SARS-CoV-2 infection. Therefore, the S protein and its subunits are being targeted by most of the strategies for vaccine design and development against SARS-CoV-2. Also, immunogenic short peptide sequences (epitopes) are being used to design and develop Multi-Epitope Vaccines (MEV). This strategy involves screening of potential epitopes from the proteome of pathogen and then to design a fusion protein by fusing the epitopes together by short peptide linkers.

The long-term adaptive immunity must involve the presentation of antigen as epitope on the surface of antigen presenting cells (APC). The proteolytic chop-down processing of antigen by proteasome and lysosome into small size peptide epitopes of different lengths, like 7, 9, 13, 15, etc. amino acids, paves the way for epitope formation and eventual presentation. The ‘Transporter associated with antigen processing’ (TAP) and further the HLA allele (human leukocyte antigen) molecules facilitate the epitope presentation. The crucial step in the process of antigen presentation is the cleavage of the antigenic protein molecules to provide small length peptides, which act as epitopes at later stage after their presentation on the surface of APC.

The different strategies as currently used to design vaccines have several limitations. Subunit based vaccines have the major limitation of not utilizing multiple target proteins/antigens of the pathogen, which is the major cause for these vaccines to have limited effect. On the other hand, the MEVs have major limitation of low possibility of the involved epitopes to remain intact and get presented in their original full sequence form by the Antigen Presenting Cells (APC) after the proteasome and/or lysosomal proteolytic processing involving cleavage of MEVs into small peptides. There have been several attempts to develop vaccine against different diseases.

U.S. Pat. No. 7,749,507 B2 describes an antigen based vaccine against malaria comprising a fusion protein derived from Plasmodium falciparum Glutamate-rich protein (GLURP) genetically coupled to at least one other Plasmodium falciparum derived protein or homologues hereof.

U.S. Pat. No. 10,183,066 B2 describes a mixture of recombinant proteins suitable as an immunogenic composition for inducing an immune response in a human against the parasite Plasmodium falciparum.

EP 1 529 536 A1 describes an immunogenic composition comprising a determined active principle and sucrose acetate isobutyrate (SAIB). These immunogenic compositions can also comprise solvents and additives such as bio-degradable polymers or adjuvants.

SARS-CoV-2 is the causal coronavirus for the COVID-19, the ongoing pandemic 2019-21. COVID-19 has caused more than 213,050,725 reported cases and more than 4,448,352 deaths worldwide (August 2021).

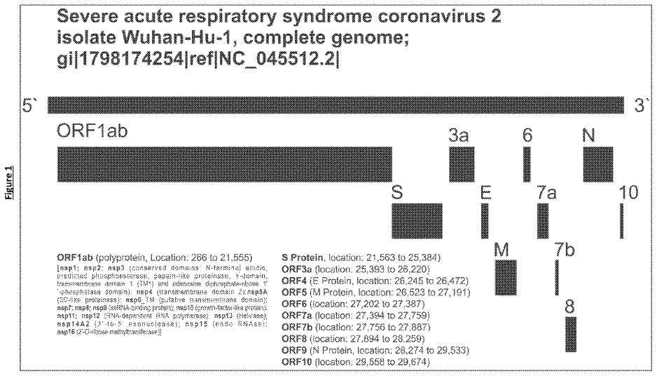

The SARS-CoV-2 proteome consists of 11 gene ORF (Open Reading Frames) expressing SARS-CoV-2 proteins. Out of these 11 ORFs, the ORF1ab is a polyprotein for 16 proteins (FIG. 1). The residual 10 ORFs express other structural and non-structural proteins that play different but essential roles for SARS-CoV-2 proliferation and pathogenesis. Thus, these structural and non-structural proteins of SARS-CoV-2 provide important targets for drug and vaccine design.

The Multi-Epitope Vaccines (MEV) strategy involves screening of potential epitopes from the proteome of SARS-CoV-2, and then to design a fusion protein by fusing the epitopes together by short peptide linkers. Subunits of different proteins of SARS-CoV-2, like the Spike protein, Envelope protein, Membrane Protein, and the Nucleocapsid Protein are being used or considered for designing and developing vaccine candidates against SARS-CoV-2.

Diagnostic kits as currently designed are also mostly based on a single or a few proteins or subunits of proteins. This strategy has the drawback that since the pathogens undergo frequent mutations, hence this may lead to a false diagnosis.

Overall, the present strategies of using single proteins or their subunits or individual epitope or multiple epitopes for vaccine and or diagnostic kit development lead to lower efficiency and insufficient specificity and could even leads to false diagnosis due frequent mutations.

It is therefore an object of the present invention to provide a new approach to the design of immunogenic compositions, in particular in case of vaccines and diagnostic kits against pathogens or other immunological targets. Other objects and advantages will become apparent to the person of skill when studying the present description of the present invention.

The following presents a simplified summary of the disclosure in order to provide a basic understanding to the reader. A more complete appreciation of the present invention and the scope thereof can be obtained from the accompanying drawings and the following detailed description of the presently preferred embodiments.

In a first aspect of the present invention, the above object is solved by a method for producing an immunogenic composition of peptide sequences from at least one proteome of interest, comprising the steps of a) providing multiple epitope sequences from said at least one proteome of interest, b) aligning the multiple epitope sequences of a) in order to generate at least one immunogenic peptide sequence comprising/consisting of an antigenic patch (Ag-Patch or epitope sequences patch) consisting of at least two overlapping epitope sequences from a region of a polypeptide from said at least one proteome of interest, c) combining said Ag-Patch into an immunogenic polypeptide construct, the construct comprising at least one Ag-Patch, at least one linker peptide sequence, and at least one adjuvant peptide sequence, and thereby producing an immunogenic composition of peptide sequences from at least one proteome of interest. Preferably, said Ag-Patches (epitope sequences patch) are identical to at 90%, preferably to at least 95% on the amino acid level between different related proteomes of interest.

The proteome of interest may be derived from cells of pathogenic or non-pathogenic organisms, such as bacteria, parasite or fungi; cancerous cells; plant cells; viruses; cells recombinantly expressing proteins; infected cells; or other source proteomes. The immunogenic composition of peptide sequences may comprise two or more, preferably three or more, or more preferably four or more Ag-Patches (epitope sequences patch).

In a second aspect of the present invention, the above object is solved by an immunogenic composition of peptide sequences, produced according to the method according to the present invention as disclosed herein.

In a third aspect of the present invention, the above object is solved by the immunogenic composition of peptide sequences according to the present invention in form of a pharmaceutical composition, a vaccine, preferably a vaccine against two or more pathogens, an anti-cancer vaccine, or a diagnostic kit.

In a fourth aspect of the present invention, the above object is solved by the immunogenic composition of peptide sequences according to the present invention, selected from the group of polypeptides consisting of SEQ ID NO: 123 to SEQ ID NO: 127.

In a fifth aspect of the present invention, the above object is solved by the therapeutic or diagnostic kit, comprising the immunogenic composition of peptide sequences according to the present invention, together with suitable auxiliary agents, packaging, and/or instructions for use, in particular an immunoprecipitation assay kit.

In a sixth aspect of the present invention, the above object is solved by the use of the immunogenic composition of peptide sequences according to the present invention or the therapeutic or diagnostic kit according to the present invention for detecting a proteome of interest, wherein preferably said proteome of interest is derived from cells of pathogenic or non-pathogenic organisms, such as bacteria, parasite or fungi; cancerous cells; plant cells; viruses, such as, for example pathogenic viruses, such as SARS-CoV-2; cells recombinantly expressing proteins; infected cells; or other source proteomes. Another aspect relates to the use of the immunogenic composition of peptide sequences according to the present invention or the therapeutic or diagnostic kit according to the present invention for identifying and/or producing a set of antibodies, T-cell receptors and/or T-cells or B-cells that are specific for a proteome of interest, wherein preferably said proteome of interest is derived from cells of pathogenic or non-pathogenic organisms, such as bacteria, parasite or fungi; cancerous cells; plant cells; viruses, such as, for example pathogenic viruses, such as SARS-CoV-2; cells recombinantly expressing proteins; infected cells; or other source proteomes.

In a seventh aspect of the present invention, the above object is solved by the immunogenic composition of peptide sequences according to the present invention or the therapeutic or diagnostic kit according to the present invention for use in the prevention and/or treatment of diseases, preferably for use in the prevention and/or treatment of a condition or disease selected from the group consisting of an infection by pathogenic or non-pathogenic organisms, such as bacteria, parasite fungi or pathogenic viruses, such as SARS-CoV-2, and cancer. This aspect also relates to a method for preventing and/or treating a condition or disease selected from the group consisting of an infection by pathogenic or non-pathogenic organisms, such as bacteria, parasite fungi or pathogenic viruses, such as SARS-CoV-2, and cancer in a subject in need thereof, comprising administering to said subject an effective amount of the immunogenic composition of peptide sequences according to the present invention.

The present invention generally relates to novel methodology for vaccine or diagnostic kit design by using antigenic patches (“Ag-Patches” or “epitope sequences patches”) identified by the inventive “overlapping-epitope-cluster-to-patches” method as described herein. The provided Multi-Patch Vaccines (MPVs) and Ag-Patch (epitope sequences patch) based diagnostic kit in the present invention provide a significant remedy for the drawbacks of the present strategies of vaccine and diagnostic kit design and developments.

In a first aspect of the present invention, the invention provides a method for producing an immunogenic composition of peptide sequences from at least one proteome of interest, comprising the steps of a) providing multiple epitope sequences from said at least one proteome of interest, b) aligning the multiple epitope sequences of a) in order to generate at least one immunogenic peptide sequence comprising/consisting of the antigenic patch (Ag-Patch or epitope sequences patch) consisting of at least two overlapping epitope sequences from a region of a polypeptide from said at least one proteome of interest, c) combining said Ag-Patch into an immunogenic polypeptide construct, the construct comprising at least one Ag-Patch, at least one linker peptide sequence, and at least one adjuvant peptide sequence, and thereby producing an immunogenic composition of peptide sequences from at least one proteome of interest.

In the context of the present invention, a proteome of interest shall refer to a collection or group of polypeptides that are encoded by the genome or other nucleic acids of a particular cell or organism, including viruses, and that are expressed at least to some extent during the life cycle of said cell or organism. The proteins constituting the proteome may also include recombinantly expressed proteins and/or proteins that are expressed by cells that are infected. Preferably, the proteome consists of proteins/polypeptides that comprise epitope sequences.

Preferred is a method according to the present invention, wherein said proteome of interest is derived from cells of pathogenic or non-pathogenic organisms, such as bacteria, parasite or fungi; cancerous cells; plant cells; viruses; cells recombinantly expressing proteins; infected cells; or other source proteomes; as well as mixtures or combinations of these.

The proteome of interest may be of reduced number, amount and/or complexity, and may include or largely or fully consist of desired and thus preselected polypeptides selected from the group of membrane- or membrane-associated polypeptides, extracellular polypeptides, non-cytosolic polypeptides, envelope polypeptides, and polypeptides that are accessible to binding by antibodies and/or T-cells. Again, also this proteome consists of proteins/polypeptides that comprise epitope sequences.

In the context of the present invention, an epitope or an epitope sequence, also known as antigenic determinant, is the part of an antigen that is recognized by the immune system, specifically by antibodies, B cells, or T cells. Epitopes can be B cell epitopes showing potential to bind or observed or found to bind antibodies, T cell epitopes showing potential to bind or observed or found to bind by T cells through T-cell receptors. T cell epitopes may be further classified into CTL (CD8+ T cell) epitopes and HTL (CD4+ T cell) epitopes (CTL: Cytotoxic T lymphocytes; HTL: Helper T lymphocyte).

Preferred is a method according to the present invention, wherein said epitope is an amino acid sequence specifically bound by an antibody or by an epitope binding fragment of said antibody, a B-cell receptor binding epitope, a T-cell receptor binding epitope or an epitope for an antigen binding fragment thereof, in particular an MHC-restricted T-cell binding epitope, a cytotoxic T-lymphocyte (CTL) binding epitope, and/or a helper T-lymphocyte (HTL) binding epitope.

Subunit vaccines according to the state of the art lag in terms of using only single or a few subunits for vaccines design, hence they lag in producing greater efficiency for immune responses. The here provided MPV design uses the Ag-Patches (epitope sequences patch) from the entire proteome, e.g. of a pathogen or group of pathogens, the MPVs are expected to produce a highly efficient immunogenic response in comparison to the subunit vaccines. The presentation of the epitopes on the APC cell surface is one of the crucial steps for T cell immune response, for which the MPVs in comparison to the MEVs provide a better option for intact epitope presentation after the proteasome and/or lysosomal processing. The Ag-Patches according to the invention provide a larger stretch of amino acid sequence with the potential to have a multiple epitope response from the epitope clusters and thus a proteolytic processing by proteasome and/or lysosome of these Ag-Patches is more probable to provide intact epitopes in comparison to the individual epitopes utilized to design MEVs.

Preferably, the immunogenic peptide sequence, that comprises/consists of the antigenic patch (Ag-Patch or epitope sequences patch) as used in method according to the present invention are identical to at least 90% or more, preferably at least 95% or more, more preferably 96% or more, 97% or more, 98% or more, 99% or more or 100% on the amino acid level between different related proteomes of interest, that is, Ag-Patch from polypeptides or proteins that are part of proteomes of or derived from cells or organisms from the same strain or species and/or from closely related strains or species, like E. coli and Salmonella, or mammalian cells etc. Related proteomes also include phenotypically or functionally related proteomes, like cells recombinantly expressing proteins or antibiotic resistant bacteria, like Methicillin-resistant Staphylococcus aureus (MRSA). Preferred is thus the method according to the present invention, wherein said epitopes are from two, three, four or more proteomes of interest, such as for example from different strains or members of the same species or genus.

The present invention uses overlapping epitope clusters to identify and assemble Ag-patches (epitope sequences patch) from, for example, the pathogen proteins. This approach is herein designated “reverse epitomics”, in view of the direction from epitopes to antigenic patches. According to a preferred embodiment of the method according to the present invention, the overlapping epitope sequence patch comprises between 2 and 20 (or more), preferably between 4 and 15 (or more), and more preferably between 5 and 12 (or more) epitopes from a region of a polypeptide from said at least one proteome of interest. See also the tables as disclosed below (Tables 1 to 3).

According to a further preferred embodiment of the method according to the present invention, the immunogenic composition of peptide sequences comprises two or more, preferably three or more, or more preferably four or more epitope sequences patches (Ag-Patches).

In general, any suitable linker can be used as the at least one linker peptide sequence, which may be selected from a short peptide linker, preferably a non-immunogenic linker. Preferred is the method according to the present invention, wherein said at least one linker peptide sequence is selected from a flexible or rigid short peptide linker, such as, for example, GGGGS (SEQ ID NO: 130) or EAAAK (SEQ ID NO: 131), respectively.

In general, any suitable adjuvant peptide sequence can be used as adjuvant in the immunogenic composition of peptide sequences according to the present invention, and is preferably positioned at the N- and/or C-terminus of the construct. Preferred is the method according to the present invention, wherein said at least one adjuvant peptide sequence is selected from a protein adjuvant, such as, example human adjuvant, β defensin 2 (GIGDPVTCLKSGAICHPVFCPRRYKQIGTCGLPGTKCCKKP) (SEQ ID NO: 128) and/or, example adjuvant, human β defensin 3 (GIINTLQKYYCRVRGGRCAVLSCLPKEEQIGKCSTRGRKCCRRKK) (SEQ ID NO: 129), and is preferably positioned at the N- and/or C-terminus of the construct.

The immunogenic composition of peptide sequences according to the present invention may further include at least one tag peptide sequence, preferably positioned at the N- and/or C-terminus of the construct. Preferred is the method according to the present invention, wherein said immunogenic composition furthermore comprises at least one tag peptide sequence, such as, for example, a six histidine tag (HHHHHH, SEQ ID NO: 132).

The peptides of the identified Ag-Patch/es (epitope sequences patch) or the immunogenic composition of peptide sequences according to the present invention or parts thereof may be synthesized or recombinantly produced. Preferred is the method according to the present invention, further comprising expressing at least one nucleic acid sequence encoding for said immunogenic composition of peptide sequences in a suitable host cell, preferably as a codon optimized nucleic acid sequence. Suitable host cells are bacteria, fungal or other non-human cells, such as CHO, insect or plant cells or other mammalian cell lines or human cell lines.

Another aspect of the present invention then relates to the immunogenic composition of peptide sequences, produced according to the present invention. Preferred is the immunogenic composition of peptide sequences according to the present invention, selected from the group of polypeptides consisting of SEQ ID NO: 123 to SEQ ID NO: 127.

Another aspect of the present invention then relates to the immunogenic composition of peptide sequences according to the present invention in form of a pharmaceutical composition, a vaccine, preferably a vaccine against two or more pathogens, an anti-cancer vaccine, or a diagnostic kit, or a vaccine or a diagnostic, such as an immunoprecipitation assay, kit.

As of present, the diagnostic kits that are designed use a single or a few proteins or subunits of proteins. Recently, epitopes are also proposed and being used to design diagnostic kit for detection of the pathogens. Although these strategies are being employed for most of the diagnostic kit designs, these strategies have several drawbacks. Frequent mutations in the pathogen genome is one of them. The mutations involving deletion and/or substitution may cause an incorrect diagnosis. Hence, the here provided use of Ag-Patches (epitope sequences patch) to design diagnostic kits provides a better option to develop diagnostic kits for long term use. The Ag-Patches are identified from the entire proteome of the pathogen; furthermore, these Ag-Patches are a longer stretch of amino acid sequences with the potential to give rise to multiple epitopes, hence diagnostic kit based on Ag-Patches will be better in efficiency as well as specificity for pathogen infection diagnosis.

Preferred is a therapeutic or diagnostic kit, comprising the immunogenic composition of peptide sequences according to the present invention, together with suitable auxiliary agents, packaging, and/or instructions for use, in particular an immunoprecipitation assay kit.

Another aspect of the immunogenic composition of peptide sequences according to the present invention relates the form of a pharmaceutical composition, i.e. the composition in an administrable form, such as a solid dosage form or a liquid formulation, such as a vaccine, preferably a vaccine against two or more pathogens, or an anti-cancer vaccine. Suitable vaccine formulations are known to the person of skill in the art. Particularly preferred is an Ag-Patch (epitope sequences patch) based vaccine, which may be designed as an ethnic population-specific vaccine, based on e.g. HLA allele genotypes and their frequencies. T-cells recognize the complex of a specific MHC molecule with a particular pathogen-derived epitope. The given epitope will elicit an immune response only in an individual that expresses the epitope binding MHC molecule. This denomination of the MHC restricted T-cell responses, and the MHC polymorphism among human population provides the basis for population coverage analysis. The MHC types are expressed at dramatically different frequencies in the different ethnicities of human population worldwide. In this way, the population coverage by an epitope-MHC pair can be determined, which functions as a basis for the design of population-specific vaccines.

Another aspect of the present invention then relates to the uses of the immunogenic composition of peptide sequences according to the present invention, the pharmaceutical composition or the therapeutic or diagnostic kit according to the present invention for detecting a proteome of interest, wherein preferably said proteome of interest is derived from cells of pathogenic or non-pathogenic organisms, such as bacteria, parasite or fungi; cancerous cells; plant cells; viruses, such as, for example pathogenic viruses, such as SARS-CoV-2; cells recombinantly expressing proteins; infected cells; or other source proteomes. The detecting may be in the context of monitoring or diagnosing or to identify a proteome of interest during a treatment or infection.

Another aspect of the present invention then relates to the uses of the immunogenic composition of peptide sequences according to the present invention, the pharmaceutical composition or the therapeutic or diagnostic kit according to the present invention for identifying and/or producing a set or collection of antibodies, T-cell receptors and/or T-cells or B-cells that are specific for a proteome of interest or a part thereof, like the reduced complexity or preselected proteome of interest as disclosed herein, wherein preferably said proteome of interest is derived from cells of pathogenic or non-pathogenic organisms, such as bacteria, parasites or fungi; cancerous cells; plant cells; viruses, such as, for example pathogenic viruses, such as SARS-CoV-2; cells recombinantly expressing proteins; infected cells; or other source proteomes.

Another aspect of the present invention then relates to the immunogenic composition of peptide sequences according to the present invention the pharmaceutical composition or the therapeutic or diagnostic kit according to the present invention for use in the prevention and/or treatment of diseases, preferably for use in the prevention and/or treatment of a condition or disease selected from the group consisting of an infection by pathogenic or non-pathogenic organisms, such as bacteria, parasites, fungi or pathogenic viruses, such as SARS-CoV-2, and cancer.

Another aspect of the present invention then relates to a method for preventing and/or treating a condition or disease selected from the group consisting of an infection by pathogenic or non-pathogenic organisms, such as bacteria, parasites, fungi or pathogenic viruses, such as SARS-CoV-2, and cancer in a subject in need thereof, comprising administering to said subject an effective amount of the pharmaceutical composition or immunogenic composition of peptide sequences according to the present invention.

Other preferred uses relate to medical (like therapeutic candidates or targets) or analytical (like therapeutic candidates or targets) applications of the Ag-Patch/es (epitope sequences patch/es) or combinations. Yet another embodiment of the present invention relates to the identification of Antigenic Patches (Ag-Patches) from pathogen proteins. Yet another embodiment of the present invention relates to the use of the identified antigenic patches (Ag-Patches) for Multi-Patch Vaccine and/or diagnostic kit design and development. Other preferred uses relate to an Ag-Patch based vaccine against any pathogen, or an Ag-Patch based combined/joint vaccine against multiple pathogens.

Other preferred uses relate to Ag-Patch (epitope sequences patch) based assay kits, for example, immunoprecipitation assay kit, Ag-Patch based diagnostic kit targeting at least one specific pathogen, and/or an Ag-Patch based diagnostic kit specific for the ‘stage of infection’ diagnosis.

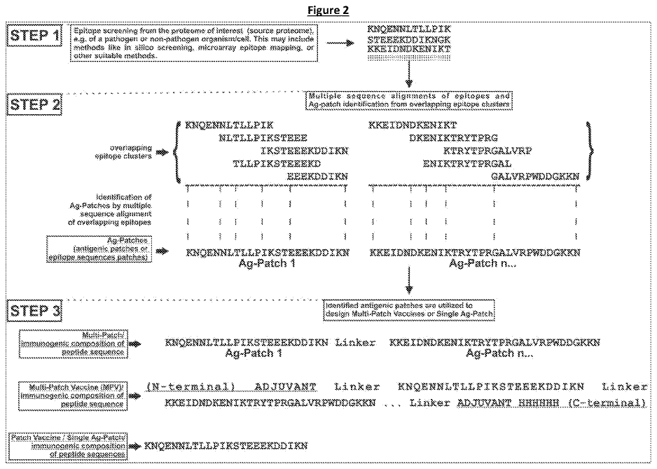

With reference to FIG. 2, providing a schematic representation of the methodology according to the present invention, the method referred herein as “overlapping-epitope-cluster-to-patches” is a stepwise method to identify highly immunogenic Ag-Patches (epitope sequences patch) from a proteome of interest, e.g. in the proteins of pathogen, that are then used to design (multi-) patch vaccines.

The first step comprises obtaining or otherwise collecting (already known) epitopes, and/or screening potential epitopes from the proteins of a proteome of interest. Known epitopes may be collected from different epitope databases or the literature. Screening of epitopes can be performed using different methods comprising in silico (like, e.g., using epitope screening tools (IEDB tools)) or in vitro methods (like, e.g., microarray epitope mapping).

The second step comprises the aligning of multiple sequences of (all) the collected epitopes. This is, for example, performed by multiple sequence alignment tools, like Clustal Omega (available at EBI server (https://www.ebi.ac.uk/Tools/msa/clustalo/). The multiple sequence alignment analysis of all the epitopes identifies clusters of overlapping epitopes, originating from a particular region of a proteome of interest, e.g. a pathogen protein, the here designated Ag-Patch or Ag-Patches (epitope sequences patch/es). The Ag-Patches are antigenic regions of a particular region of a proteome of interest, e.g. a pathogen protein, which are further used for the product design of, for example, a vaccine against the pathogen.

The third step comprises the actual design of an immunogenic composition, in particular an Ag-Patch vaccine or multi-patch vaccine (MPV) by using the antigenic patches (Ag-Patches or epitope sequences patches) identified in the second step. To design the MPV, the identified multiple Ag-Patches are fused together by short peptide linkers, like GGGGS (SEQ ID NO: 130) or EAAAK (SEQ ID NO: 131), or other suitable linkers. A preferable design schematically consists of:

(N-terminal) Ag-PATCH 1 LINKER Ag-PATCH 2 LINKER Ag-PATCH 3 LINKER Ag-PATCH 4 LINKER Ag-PATCH n (C-terminal).

This design of MPV may also be fused or complexed with at least one protein adjuvant at the N- and/or C-terminal end of the MPV, and optionally an amino acid tag, like 6×histidine tag at the C-terminal end, such as:
(N-terminal) ADJUVANT LINKER Ag-PATCH 1 LINKER Ag-PATCH 2 LINKER Ag-PATCH 3 LINKER Ag-PATCH 4 LINKER Ag-PATCH n LINKER ADJUVANT HHHHHH (C-terminal).

The Ag-patches (epitope sequences patches) as identified herein above may also be used to design a diagnostic kit for, e.g., pathogen infection diagnosis. The peptide of the identified Ag-Patch/es may be synthesized or recombinantly produced, and immobilized on a solid carrier or surface. The immobilized antigenic Patches (Ag-Patches) are used to detect the presence of antibodies against the pathogen protein in a diagnostic sample, like a patient serum sample by the anti-human secondary antibodies.

The method disclosed in the present invention identifies highly immunogenic Ag-Patches (antigenic patches or epitope sequences patch) from different proteins of the pathogen. These Ag-patches are the antigenic regions of proteins that give rise to multiple overlapping epitopes in clusters. Hence, these Ag-patches are of great significance to be used for diagnostic kits as well as (Multi-) Patch Vaccine designs. The antigenic Patches (Ag-Patches) have a greater chance to release multiple epitopes upon proteasome/lysosomal proteolytic processing in professional or non-professional antigen presenting cells, in comparison to the individual epitope or Multi-Epitope Vaccines as described herein. Also, the MPVs as prepared by the Ag-Patches identified from the entire proteome or multiple proteins of the pathogen (ideally covering most of the pathogen proteome, see above) are advantageous over to the single protein or subunit based vaccines.

For diagnostic kits, at the industrial scale, peptides for the identified Ag-Patches (epitope sequences patch) can be synthesized conveniently, compared to whole protein synthesis, and immobilized on a solid surface to develop diagnostic kits; hence the overall cost of diagnostic chip prepared by utilizing the Ag-Patches are reduced. Moreover, the Ag-Patches have longer shelf life in comparison to the full/whole protein-based conventional diagnostic kits.

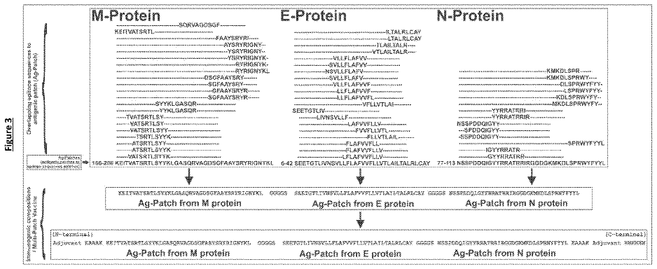

Yet another embodiment of the present invention relates to a Multi-Patch Vaccine and/or diagnostic kit designed against SARS-CoV-2, such as given in the tables herein below (Table 3). The design consists of 3 CTL Multi-Patch Vaccine and 2 HTL Multi-Patch vaccine candidates. With respect to FIGS. 2 and 3, the first step was the study of reported epitopes from the entire proteome of the SARS-CoV-2, collected from the existing literature. These epitopes were studied and analyzed in order to identify Ag-Patches (epitope sequences patch).

In the second step the collected epitopes are analyzed by the Multiple Sequence Alignment (MSA) of the epitopes performed by clustal omega tool available at the EBI server (https://www.ebi.ac.uk/Tools/msa/clustalo/). The MSA analysis resulted in overlapping epitopes to form overlapping epitope clusters (FIG. 3). These overlapping epitopes were obtained from a certain region of the protein. This region is thus recognized as antigenic region, termed here as epitope sequences patch/es or Antigenic Patch/es (Ag-Patch or Ag-Patches) (Table 1 to 2). These patches are highly immunogenic in nature, since large numbers of epitope are observed to be arising from these regions of, for example, the SARS-CoV-2 proteins.

Further, as step 3, these highly antigenic regions of the SARS-CoV-2 proteins were used to design Multi-Patch Vaccine candidate (Table 3). The identified patches were fused together by short peptide linker GGGGS (SEQID NO: 130). The fused Multi-Patch Vaccine construct was further fused with protein adjuvants, human β defensin 2 (GIGDPVTCLKSGAICHPVFCPRRYKQIGTCGLPGTKCCKKP, SEQ ID NO: 128) at N terminal and human β defensin 3 (GIINTLQKYYCRVRGGRCAVLSCLPKEEQIGKCSTRGRKCCRRKK, SEQ ID NO: 129) at C terminal of the construct as shown in Table 3. The adjuvants were fused to the vaccine construct by utilizing the short peptide linker EAAAK (SEQ ID NO: 131). In the Multi-Patch Vaccine design, a six histidine tag (HHHHHH, SEQ ID NO: 132) was also fused at the C terminal in order to facilitate the easy purification of the Multi-Patch Vaccine protein after expression in vitro or in a recombinant cell.

A preferred embodiment of the present invention relates to the identification of 73 CTL (Cytotoxic T-Lymphocyte), 49 HTL (Helper T-Lymphocyte) Ag-Patches (epitope sequences patches) from the SARS-CoV-2 proteome of interest. These antigenic patches have been identified on the basis of overlapping epitope clusters by the present method (FIG. 2). The identified patches provided are highly conserved in nature, as these patches were observed in most of the SARS-CoV-2 protein sequences available at NCBI protein sequence database (Table 1 and 2). The identified Ag-Patches give rise to a large number of epitopes, hence they are expected to be an excellent candidate for Multi-Patch Vaccine design and development. Additionally the identified Ag-Patches, due to their high immunogenicity, are potential candidates to design diagnostic kit against SARS-CoV-2 for rapid diagnosis. The provided design of an MPV was developed by fusing all the identified patches by GGGGS linkers. Furthermore, the MPV construct designed is fused to adjuvant proteins, human β defensing 2 and human β defensing 3 at N- and C-terminus of the MPV constructs as shown in Table 3. The short peptide linker EAAAK was used to fuse the adjuvant proteins.

As of yet the vaccines have been designed on the basis of subunits of SARS-CoV-2 proteins or epitopes derived from the SARS-CoV-2 proteins. The present invention provides a novel approach to identify and utilize antigenic PATCHES (epitope sequences patch) to design novel Multi-Patch Vaccines against SARS-CoV-2. Hence, the invention is very significant in terms of providing novel approach to design vaccine as well as in providing novel design of Multi-Patch Vaccine against SARS-CoV-2 infection. Furthermore, the identified antigenic patches also have great applicability to design and develop diagnostic kit against SARS-CoV-2 infection.

The identified novel antigenic patches (Ag-Patches or epitope sequences patches) in the present invention have great industrial applicability. The identified antigenic patches have applicability in both Multi-Patch Vaccine designing as well as in diagnostic kit designing. The Multi-Patch Vaccine designs provided in the present invention can be tried in vivo and after successful trials these MPVs can be industrially prepared and provided as vaccine candidate against SARS-CoVe-2 infection. Furthermore, the identified antigenic patches are used to design and develop diagnostic kits which again, after successful trial, will have great industrial application for large scale production.

The majority of the vaccines designed against pathogens, such as SARS-CoV-2, are focused on a single protein, protein subunits or the “popular” epitopes from SARS-CoV-2 proteins, mostly S, E, M, N and ORF1 ab proteins. The recent strategies to design and develop vaccine to combat SARS-CoV-2 involve subunit-vaccines or multi-epitope vaccines. The subunit vaccines involve the use of single proteins or multiple subunits of SARS-CoV-2 proteins. Multi-epitope vaccines involve the fusion of multiple epitopes as identified from the proteome of the SARS-CoV2, fused by short peptide linkers. Nevertheless, although the major focus is on epitope- or subunit-based vaccine design to combat SARS-CoV-2, both the approaches have several limitations. The major limitation with single protein or multiple subunit-based vaccine is the limited efficiency of the vaccines. Also, the epitope-based vaccines suffer from the major drawback of frequent mutations in the proteome of the SARS-CoV-2 virus. The multi-epitope-based vaccines suffer from a major challenge with respect to the presentation of chosen epitopes by the APC. The proteolytic chop down processing by proteasome and lysosome leaves a very narrow chance for the epitopes of the multi-epitope vaccines, to remain intact and to be successfully presented by APC.

The present invention relates to a novel method to design a vaccine against pathogens, such as SARS-CoV-2, by using multiple antigenic patches (Ag-Patches or epitope sequences patches) from the viral proteins. The Ag-Patches as used are identified by clusters of overlapping epitopes. As an example, the identification of these Ag-Patches was performed by reverse epitomics analysis of high scoring CTL and HTL epitopes screened from all the ORF proteins of the SARS-CoV-2 virus. All the screened epitopes were well characterized for their conservancy, immunogenicity, nontoxicity and large population coverage. The clusters of the overlapping epitopes led to the identification of Ag-Patches. These Ag-Patches from all the ORF proteins of the SARS-CoV-2 proteome were the used further to design MPV candidate vaccines against the SARS-CoV-2 infection. The designed MPVs from the antigenic patches, and exemplary of SARSCoV-2 proteins, have several advantages over to the subunit and multi-epitope-based vaccines. The Ag-Patches utilized were identified and collected from the entire proteome of the SARS-CoV-2. This enhances the efficiency of the vaccines and makes the vaccine more effective. The MPVs consisting of the identified Ag-Patches have the potential to raise multiple epitopes in clusters upon the chop-down processing by proteasome and lysosome in the APC. The identified Ag-Patches also have a higher chance that the epitopes raised after proteasome and lysosomal processing get presented by the APC and elicit an effective immune response. Since the Ag-Patches were identified from a large number of epitopes forming clusters, the MPVs designed have the potential to raise a larger number of epitopes upon proteasome and lysosomal processing; hence, a larger number of HLA alleles is targeted and hence, larger ethnic human populations are covered by the MPVs, in comparison to the limited number of epitopes used in multi-epitope vaccines.

As an example, the five MPVs designed in this invention used the Ag-Patches (antigenic patches or epitope sequences patches) that were identified by 768 (518 CTL and 250 HTL) overlapping epitopes targeting different HLA alleles. Such an inclusion (coverage) of large numbers of epitopes and targeting large numbers of HLA alleles is not possible for multi-epitope-based vaccines prepared with limited number of epitopes. All the identified Ag-Patches used to design MPVs have shown to be highly conserved amongst the protein sequences of SARS-CoV-2 as available at the NCBI protein database. All the physicochemical properties of MPVs designed against SARS-CoV-2 favor their overexpression in vitro. This is further supported by the cDNA analysis of the codon-optimized constructs of all the MPVs for high expression in a mammalian cell line (human). Furthermore, the designed MPVs also show stable binding tendency with the ectodomain of human Toll-like Receptor 3 (TLR-3(ECD)), which is an important criteria for an antigen to be recognized and processed by the human immune system.

This invention has identified highly immunogenic novel Ag-Patches (antigenic patches or epitope sequences patches) (73 CTL and 49 HTL) from the entire proteome of SARS COV-2. The Ag-Patches were identified by a novel reverse epitomics approach, the ‘overlapping-epitope-clusters-to-patches’ method. The Ag-Patches are highly conserved in nature and found in most of the SARS-CoV-2 protein sequences available in the NCBI protein database. The Ag-Patches were identified on the basis of high scoring, immunogenic, overlapping epitopes that were thoroughly screened from the entire proteome of SARS-CoV-2. Further, for the first time the inventors have used the multiple immunogenic Ag-Patches as identified to design novel MPVs, which constitutes a new methodology for vaccine design. The MPVs designed against SARS-CoV-2 in the invention have potential to give rise to a total of 768 epitopes (518 CTL and 250 HTL epitopes) targeting a large number of different HLA alleles. Such a large number of epitopes cannot be used (covered) in multi-epitope-based vaccines. The large number of epitopes as covered causes a large number of HLA alleles to be targeted, further implying large ethnic human population coverage worldwide. The MPVs with multiple epitope cluster based Ag-Patches, in this case from the entire proteome of SARS-CoV-2, have potential to provide larger number of epitopes in comparison with the MEVs upon proteasome or lysosomal chop down processing by the APC. The designed MPVs against SARS-CoV-2 were validated for stable complex formation with the ectodomain of TLR-3. The physiochemical properties and the codon-optimized cDNA analysis of all the MPVs designed suggests a favored large-scale expression potential. The inventors conclude that the novel MPVs as designed from the novel Ag-Patches have a high potential to combat infections, such as SARS-CoV-2, with greater effectiveness, high specificity, and large human population coverage worldwide.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The present invention will now be described further in the following examples with reference to the accompanying Figures and the sequence listing, nevertheless, without being limited thereto. For the purposes of the present invention, all references as cited herein are incorporated by reference in their entireties. In the Figures,

FIG. 1 shows a schematic representation of all ORFs of proteins as expressed by the SARS-CoV-2 genome in accordance with an exemplary embodiment of the present disclosure.

FIG. 2 shows a schematic flowchart for the methodology of the invention called “overlapping-epitope-clusters-to-patches” in accordance with an exemplary embodiment of the present disclosure. Sequences as shown are: STEEEKDDIKNGK (SEQ ID NO: 133), KNQENNLTLLPIK (SEQ ID NO: 134), NLTLLPIKSTEEE (SEQ ID NO: 135), IKSTEEEKDDIKN (SEQ ID NO: 136), TLLPIKSTEEEKD (SEQ ID NO: 137), EEEKDDIKN (SEQ ID NO: 138), KNQENNLTLLPIKSTEEEKDDIKN (SEQ ID NO: 139), KKEIDNDKENIKT (SEQ ID NO: 140), DKENIKTRYTPRG (SEQ ID NO: 141), KTRYTPRGALVRP (SEQ ID NO: 142), ENIKTRYTPRGAL (SEQ ID NO: 143), GALVRPWDDGKKN (SEQ ID NO: 144), KKEIDNDKENIKTRYTPRGALVRPWDDGKKN (SEQ ID NO: 145), and HHHHHH (SEQ ID NO: 132).

FIG. 3 shows the exemplary identification of Ag-Patches (antigenic patches or epitope sequences patches) from three proteins (Membrane protein (M), Envelope protein (E) and the Nucleocapsid protein (N)) of SARS-CoV-2, in accordance with a preferred embodiment of the present disclosure, and a design of the inventive Multi-Patch Vaccine by utilizing the identified antigenic patches (Ag-Patches) from M protein, E protein and N protein of the SARS-CoV-2 in accordance with a preferred embodiment of the present disclosure. Sequences as shown are: SQRVAGDSGF (SEQ ID NO: 146), KEITVATSRTL (SEQ ID NO: 147), FAAYSRYRI (SEQ ID NO: 148), AYSRYRIGNY (SEQ ID NO: 149), YSRYRIGNY (SEQ ID NO: 150), YSRYRIGNYK (SEQ ID NO: 151), RYRIGNYK (SEQ ID NO: 152), RYRIGNYKL (SEQ ID NO: 153), DSGFAAYSRY (SEQ ID NO: 154), SGFAAYSRY (SEQ ID NO: 155), GFAAYSRYR (SEQ ID NO: 156), SGFAAYSRYR (SEQ ID NO: 157), SYYKLGASQR (SEQ ID NO: 158), YYKLGASQR (SEQ ID NO: 159), TVATSRTLSY (SEQ ID NO: 160), VATSRTLSY (SEQ ID NO: 161), VATSRTLSYY (SEQ ID NO: 162), TSRTLSYYK (SEQ ID NO: 163), ATSRTLSYY (SEQ ID NO: 164), ATSRTLSYYK (SEQ ID NO: 165), VATSRTLSYYK (SEQ ID NO: 166), KEITVATSRTLSYYKLGASQRVAGDSGFAAYSRYRIGNYKL ((SEQ ID NO: 3), (SEQ ID NO: 167) to (SEQ ID NO: 186), ILTALRLCAY (SEQ ID NO: 167), LTALRLCAY (SEQ ID NO: 168), TLAILTALR (SEQ ID NO: 169), VTLAILTALR (SEQ ID NO: 170), VLLFLAFVV (SEQ ID NO: 171), SVLLFLAFVV (SEQ ID NO: 172), NSVLLFLAFV (SEQ ID NO: 173), SVLLFLAFV (SEQ ID NO: 174), VLLFLAFVVF (SEQ ID NO: 175), LLFLAFVVF (SEQ ID NO: 176), LLFLAFVVFL (SEQ ID NO: 177), VFLLVTLAI (SEQ ID NO: 178), SEETGTLIV (SEQ ID NO: 179), LIVNSVLLF (SEQ ID NO: 180), LAFVVFLLV (SEQ ID NO: 181), FVVFLLVTL (SEQ ID NO: 182), FLLVTLAIL (SEQ ID NO: 183), FLAFVVFLL (SEQ ID NO: 184), FLAFVVFLLV (SEQ ID NO: 185), LFLAFVVFLLV (SEQ ID NO: 186), SEETGTLIVNSVLLFLAFVVFLLVTLAILTALRLCAY (SEQ ID NO: 55), KMKDLSPR (SEQ ID NO: 187), KMKDLSPRWY (SEQ ID NO: 188), DLSPRWYFYY (SEQ ID NO: 189), LSPRWYFYY (SEQ ID NO: 190), KDLSPRWYFY (SEQ ID NO: 191), MKDLSPRWYFY (SEQ ID NO: 192), YYRRATRRI (SEQ ID NO: 193), YYRRATRRIR (SEQ ID NO: 194), NSSPDDQIGYY (SEQ ID NO: 195), SPDDQIGYY (SEQ ID NO: 196), SSPDDQIGYY (SEQ ID NO: 197), SPRWYFYYL (SEQ ID NO: 198), IGYYRRATR (SEQ ID NO: 199), GYYRRATRR (SEQ ID NO: 200), NSSPDDQIGYYRRATRRIRGGDGKMKDLSPRWYFYYL (SEQ ID NO: 29), GGGGS (SEQ ID NO: 130), EAAAK (SEQ ID NO: 131) and HHHHHH (SEQ ID NO: 132).

### EXAMPLES

### Supporting Study on SARS-CoV-2

While the present examples are relating to potentially immunogenic Ag-Patches (antigenic patches or epitope sequences patches) from the SARS-CoV-2 proteome, the person of skill will be readily able to adjust the methods and uses as described to other proteomes and pathogenic organisms, such as bacteria, fungi, cancerous cells or other source proteomes for the generation of immunogenic compositions according to the present invention.

### Methods and Results

Antigenic patches (Ag-Patch) or the epitope sequence patch from all the eleven ORF proteins of SARS-CoV-2 proteome (NCBI: SARS-CoV-2 isolate Wuhan-Hu-1, complete genome; gij1798174254jrefjNC_045512.2j) were identified by the overlapping epitope clusters. To identify the AgPatches from the SARS-CoV-2 proteins, first high scoring CTL and HTL epitopes from all the eleven ORF proteins of the virus were screened. Further, Multiple Sequence Alignment (MSA) was performed for all the screened epitopes, leading to clusters of overlapping epitopes and determination of CTL and HTL Ag-Patches. The overlapping epitopes forming clusters and their HLA allele binders were used to analyze world population coverage. Further, three CTL and two HTL Multi-Patch Vaccines (MPVs) were designed by the Ag-Patches, Human β defensin 2 and 3 (as adjuvants) and short peptide linkers GGGGS (SEQ ID NO: 130) and EAAAK (SEQ ID NO: 131). Next, the CTL and HTL MPV models were validated for their molecular interaction with the ectodomain of the Toll-Like Receptor 3. Further, the codon optimized cDNA for all CTL and HTL MPVs were analyzed and found to favor high expression in human cell line.

Screening of Potential Epitopes from the SARS-CoV2 Proteome

### Screening of CTL Epitopes

The screening of CTL epitopes was performed using the IEDB (Immune Epitope Database) tools ‘MHC-I Binding Predictions’ (http://tools.iedb.org/mhci/) and ‘MHC-I Processing Predictions’. The tools generate ‘Percentile rank’ and a ‘total score’, respectively, indicating the immunogenic potential of the screened epitopes. Immunogenicity of all the screened CTL epitopes was also obtained by using the ‘MHC I Immunogenicity’ tool of IEDB with all the parameters set to default analyzing 1st, 2nd and C-terminal amino acids of the given epitope.

A total of 1013 CTL epitopes and HLA I alleles pairs were screened. The high immunogenicity score of all the screened CTL epitopes was observed which suggests the high immunogenic potential of the CTL epitopes.

### Screening of HTL Epitopes.

To screen out the HTL epitopes from SARS-CoV-2 proteins, the IEDB tool ‘MHC-II Binding Predictions’ was used. The tool generates a ‘Percentile Rank’ for each potential screened peptide. A total of 314 HTL epitopes-HLA II allele pairs with high percentile rank were screened from the entire proteome of the SARS-CoV-2 virus.

### CTL and HTL Epitope Toxicity Prediction

The tool ToxinPred was used to analyze the toxicity of screened CTL and HTL epitopes. The tool identifies highly toxic or nontoxic short peptides. The toxicity check analysis was done by the ‘SVM (Swiss-Prot) based’ (support vector machine) method.

The ToxinPred study of all the screened CTL and HTL epitopes revealed that all the screened epitopes were non-toxic.

### From Overlapping CTL and HTL Epitope Clusters to Ag-Patches

### Overlapping CTL and HTL Epitope Cluster Based Ag-Patch Identification

To identify the potentially immunogenic Ag-Patches from SARS-CoV-2 proteome, all screened high-scoring epitopes were aligned against their respective source protein sequence by MSA tool Clustal Omega available on the EBI server. The amino acid sequence patches of the SARS-CoV-2 protein sequences showing consensus with the clusters of overlapping epitopes were chosen and shortlisted as Ag-Patches (antigenic patches or epitope sequence patch). This approach of search and identification of antigenic patches from source protein in a reverse epitomics manner, i.e. from epitopes to antigenic patches of source protein, is herein defined as ‘overlapping-epitope-clusters-to-patches’ method (see FIG. 2).

A total of 73 Ag-Patches from CTL and a total of 49 Ag-Patches from HTL high-scoring 768 overlapping epitopes (518 CTL and 250 HTL epitopes) were identified (Tables 1 to 2) using the ‘Overlapping-epitope-clusters-to-patches’ method (FIG. 2). The Ag-Patches thus obtained from the CTL and HTL epitope clusters are expected to produce clusters of up to 768 overlapping epitopes, targeting different HLA allele, upon proteolytic chop down processing by professional and non-professional antigen presenting cell (APC). This is a crucial step for the MPVs and shows a strong possibility for the epitopes to be processed into their intact form and presented by APC. On the other hand, such large numbers of epitopes are not possible to be accommodated by the MEVs. The MEVs would also face challenges to give raise to the epitopes in their intact form upon the said proteolytic chop down processing by APC.

### Population Coverage by the Overlapping CTL and HTL Epitopes

The ‘Population Coverage’ tool of IEDB was used to analyze the world human population coverage for both the CTL and HTL overlapping epitopes and their respective HLA allele-binding pairs.

Ag-Patches as identified cover 518 CTL and 250 HTL overlapping epitopes targeting 27 HLA class I and 28 HLA class II alleles, respectively. This leads to large human population coverage of 99.98% (average being 91.11 and standard deviation being 16.97). In the inventors' analysis, the complete human population ethnicity distribution worldwide was included. The countries most affected by COVID-19 show significant coverage.

### Conservation Analysis of Antigenic Patches

The shortlisted CTL and HTL epitope cluster based Ag-Patches identified from 11 SARS-CoV-2 ORF proteins were further analyzed for their amino acid sequence conservation by the ‘Epitope Conservancy Analysis’ tool of IEDB. The epitope conservancy is the percentage of SARS-CoV-2 ORF protein sequences (retrieved from NCBI) containing the particular epitope cluster-based Ag-Patch with 100% amino acid sequence match.

The inventors found that all the identified immunogenic CTL and HTL Ag-Patches are significantly conserved with a conservancy range of 91.23% to 100% with only four out of total 122 Ag-Patches with conservancy of 77.33%, 67.33%, 51.25%, 47.50%.

### Design of Multi-Patch Vaccines MPVs

The identified overlapping epitope cluster based Ag-Patches from the proteome of the SARS-CoV-2 were used to design three CTL and two HTL MPVs. The short-peptide linkers EAAAK (SEQ ID NO: 131) and GGGGS (SEQ ID NO: 130) were used as rigid and flexible linkers, respectively. The short-peptide linker EAAAK (SEQ ID NO: 131) facilitates the domain formation and provides a rigid link between two domains facilitating the protein to fold in a stable tertiary conformation. The short and flexible peptide linker GGGGS (SEQ ID NO: 130) provides conformational flexibility and hence facilitates stable conformation to the final folded protein structure. The GGGGS (SEQ ID NO: 130) was used to fuse the Ag-Patches together, and to ease folding of the protein into its tertiary conformation. The rigid linker EAAAK (SEQ ID NO: 131) was used to fuse the human β defensin 2 and 3 (hBD-2 and hBD-3) at N and C terminal of the MPVs, respectively. The human β defensin 2 and 3 were used here as an adjuvant to enhance immunogenic response.

### Physicochemical Property Analysis of Designed MPVs

The empirical physicochemical properties of the amino acid sequences of the designed three CTL and two HTL MPVs were analyzed by the ProtParam tool.

The molecular weight of all the MPVs ranges from 66.36 to 89.96 kDa. The expected half-life of up to 30 h in mammalian cells is very favorable for all the MPVs for expression and purification in vitro. The aliphatic index (53.51 to 100.86) and grand average of hydropathicity (GRAVY) (−0.274 to 0.445) of all the MPVs indicate their globular and hydrophilic nature. The instability index score of all the MPVs (39.68 to 53.37) indicates the stable nature of the protein molecules upon expression in vitro. Overall, the physiochemical parameters of all MPVs suggest a favorable expression of MPVs in vitro.

### MPVs Allergenicity and Antigenicity Prediction

All the designed CTL and HTL MPVs were further analyzed for allergenicity and antigenicity prediction by utilizing the AlgPred and Vaxigen tools, respectively.

All three CTL and two HTL MPVs were suggested as non-allergen, (scoring: 0.46602545 for CTL-MPV-1; 0.71579187 for CTL-MPV-2; 0.90796056 for CTL-MPV-3; 0.56197384 for HTL-MPV-1 and 0.72617142 for HTL-MPV2; while the threshold cutoff of 0.4). Similarly, all three CTL and two HTL MPVs were found to be potential antigens as suggested by Vaxijen analysis (scoring: 0.5241 for CTL-MPV-1, 0.4811 for CTL-MPV-2, 0.6534 for CTL-MPV-3, 0.5016 for HTL-MPV-1 and 0.5096 for HTL-MPV-2; default threshold being 0.4). Largely, AlgPred and VaxiJen analysis of all the three CTL MPVs and two HTL MPVs suggested that all the MPVs are non-allergic in nature, but antigenic.

### Tertiary Structure Modelling and Refinement of MPVs

The tertiary structure of all the designed three CTL and two HTL MPVs were generated by homology modelling utilizing the ITASSER modelling tool. The I-TASSER is a tool that uses the sequence-to-structure-to-function paradigm for protein structure prediction. Further, The refinement of all the generated three CTL and two HTL MPV models were performed by ModRefiner and GalaxyRefine tools. The models with highest scoring for TM-Score, MolProbity, etc. were chosen for further studies.

### Validation of CTL and HTL MPVs Refined Models

The refined three CTL and two HTL MPV tertiary models were observed to have acceptable conformations as validated by Ramachandran plot study by RAMPAGE.

### Molecular Interaction Analysis of MPVs and Immune Receptor

The inventors tested all the CTL and HTL MPV models for their molecular interaction with the ectodomain (ECD) of the TLR3 receptor by molecular docking and MD simulation studies. Protein-protein molecular docking was performed by the tool PatchDock. The PatchDock tool utilizes an algorithm for unbound (mimicking real-world environment) docking of molecules for protein-protein complex formation. For molecular docking, the X-ray crystal structure of human TLR3 ectodomain (ECD) was retrieved from PDB databank (PDB ID: 2A0Z). The MPVs-TLR3(ECD) complex molecular interactions were further evaluated using MD simulation analysis by using the YASARA tool (Yet Another Scientific Artificial Reality Application). The MD simulations studies were carried out in an explicit water environment in a dodecahedron simulation box at a stabilized temperature of 298K, pressure of 1 atm and pH 7.4, with periodic cell boundary condition. The solvated systems were neutralized with counter ions (NaCl) (concentration 0.9M). The AMBER14 force field was used on the systems during MD simulation. The long-range electrostatic energy and forces were calculated using particle mesh-based Ewald method. The solvated structures were energy minimized by the steepest descent method at a temperature of 298K and a stable pressure of 1 atm. Further, the complexes were equilibrated for period of Ins. After equilibration, a production MD simulation was run for 20 nanoseconds at a stable temperature and pressure and time-frames were saved at every 10 ps, for each MD simulations.

The generated docking complex conformations with the highest docking score were chosen for further study [CTL-MPV-1:TLR3(ECD) (docking score: 17696), CTL-MPV-2:TLR3(ECD) (docking score: 17118), CTL-MPV-3:TLR3(ECD) (docking score: 16562), HTLMPV-1:TLR3(ECD) (docking score: 21432) and HTL-MPV-2:TLR3(ECD) (docking score: 17620)]. The highest docking score indicates the MPV and TLR3(ECD) complexes to have best geometric shape complementarity fitting conformation. Furthermore, the molecular docking and MD simulation study of all the MPVs and TLR3(ECD) complexes suggests a stable complex formation tendency for all the MPVs with TLR3(ECD) with stable RMSD and RMSF values for Ca, Backbone and all the atoms of all the CTL and HTL MPVs.

Analysis of MPVs cDNA for Expression in Human Host Cell Line.

Codon-optimized complementary DNA (cDNA) of all the three CTL and two HTL MPVs were generated for favored expression in Mammalian cell line (Human) by Java Codon Adaptation Tool. The generated cDNA of all the MPVs was further analyzed by GenScript Rare Codon Analysis Tool for its large-scale expression potential.

The analysis revealed that the codon optimized cDNA of all the CTL and HTL MPVs satisfy all the crucial parameters such as GC content, CAI (Codon Adaptation Index) score and 0% tandem rare codons for high-level expression in a mammalian cell line (human). Hence, the cDNA of all the MPVs has a high potential for large-scale expression in the human cell line.

### Example 1

Identification of three immunogenic Ag-Patches (antigenic patches or epitope sequences patches) from the proteins of SARS-CoV-2 using the overlapping-epitope-clusters-to-patches method according to the invention (see FIGS. 2 and 3).

Step 1: Multiple epitope sequences from the membrane protein (M), envelope protein (E) and the nucleocapsid protein (N) of SARS-CoV-2 were collected from the literature (see Grifoni, A., et al. (2020), Srivastava, S., Verma, S., Kamthania, M., Kaur, R. et al. (2020), Srivastava, S., Verma, S., Kamthania, M., Agarwal, D. et al. (2020).

Step 2: The thus collected epitope sequences were analyzed using multiple sequence alignment by clustal omega tool available at the EBI server. Several overlapping epitope clusters were identified.

The same approach was used in order to identify overlapping epitope cluster based CTL patches from the entire proteome of the SARS-CoV-2, as listed in Tables 1 to 3.

TABLE 1
Overlapping epitope cluster based CTL Ag-Patches (antigenic patches or epitope
sequences patches) derived from the entire proteome of the SARS-CoV-2 virus.
The highly immunogenic patches as identified were used to
design three CTL Multi-Patch Vaccines, called CTL-MPV-1, CTL-MPV-2 and
CTL-MPV-3. The patches as identified were also highly conserved in nature.

| | | | # | | Source |
|---|---|---|---|---|---|
| Seq. | | | epitopes | | SARS- |
| Id | CTL- | CTL epitope cluster based patch | in | | CoV-2 |
| No. | MPVs | sequence | cluster | Conserved | protein |
| 1 | CTL- | GTITVEELKKLLEQWNLVIGFLFLTWI | 36 | 96.64% | M- |
| | MPV-1 | CLLQFAYANRNRFLYIIKLIFLWLLWP | | (461/477) | Protein/6- |
| | | VTLACFVLAAV | | | 70 |
| 2 | CTL- | RINWITGGIAIAMACLVGLMWLSYFI | 28 | 96.01% | M- |
| | MPV-1 | ASFRLFARTRSMWSFN | | (458/477) | Protein/72- |
| | | | | | 113 |
| 3 | CTL- | KEITVATSRTLSYYKLGASQRVAGDS | 21 | 97.69% | M- |
| | MPV-1 | GFAAYSRYRIGNYKL | | (466/477) | Protein/166 |
| | | | | | -206 |
| 4 | CTL- | TTRTQLPPAYTNSFTRGVYYPDKVFR | 6 | 93.02% | S- |
| | MPV-1 | SSV | | (467/502) | Protein/19- |
| | | | | | 47 |
| 5 | CTL- | STQDLFLPFFSNVTWF | 4 | 91.43% | S- |
| | MPV-1 | | | (459/502) | Protein/50- |
| | | | | | 65 |
| 6 | CTL- | VLPFNDGVYFASTEKSNIIRGWIF | 6 | 93.62% | S- |
| | MPV-1 | | | (470/502) | Protein/83- |
| | | | | | 106 |
| 7 | CTL- | GVYYHKNNKSWMESEFRVYSSANNC | 7 | 91.23% | S- |
| | MPV-1 | TFEYVSQPFL | | (458/502) | Protein/142 |
| | | | | | -176 |
| 8 | CTL- | KQGNFKNLREFVFKNIDGYFKIYSKH | 4 | 91.83% | S- |
| | MPV-1 | TPI | | (461/502) | Protein/182 |
| | | | | | -210 |
| 9 | CTL- | EPLVDLPIGINITRFQTLLA | 3 | 93.22% | S- |
| | MPV-1 | | | (468/502) | Protein/224 |
| | | | | | -243 |
| 10 | CTL- | TPGDSSSGWTAGAAAYYVGYLQPRT | 6 | 93.42% | S- |
| | MPV-1 | FLLK | | (469/502) | Protein/250 |
| | | | | | -278 |
| 11 | CTL- | SETKCTLKSFTVEKGIYQTSNFR | 4 | 93.22% | S- |
| | MPV-1 | | | (468/502) | Protein/297 |
| | | | | | -319 |
| 12 | CTL- | FPNITNLCPFGEVENATRFASV | 3 | 93.42% | S- |
| | MPV-1 | | | (469/502) | Protein/329 |
| | | | | | -350 |
| 13 | CTL- | RISNCVADYSVLYNSASFSTFKCY | 7 | 93.82% | S- |
| | MPV-1 | | | (471/502) | Protein/357 |
| | | | | | -380 |
| 14 | CTL- | NVYADSFVIRGDEVR | 2 | 93.62% | S- |
| | MPV-1 | | | (470/502) | Protein/394 |
| | | | | | -408 |
| 15 | CTL- | SKVGGNYNYLYRLFRKSNLKPFER | 10 | 92.62% | S- |
| | MPV-1 | | | (465/502) | Protein/443 |
| | | | | | -466 |
| 16 | CTL- | NTSNQVAVLYQDVNCTEV | 3 | 67.33% | S- |
| | MPV-1 | | | (338/502) | Protein/603 |
| | | | | | -620 |
| 17 | CTL- | RVYSTGSNVF | 2 | 93.82% | S- |
| | MPV-1 | | | (471/502) | Protein/634 |
| | | | | | -643 |
| 18 | CTL- | SPRRARSVASQSIIAY | 5 | 93.82% | S- |
| | MPV-1 | | | (471/502) | Protein/680 |
| | | | | | -695 |
| 19 | CTL- | FTISVTTEILPVSMTKTSVDCTMY | 4 | 93.82% | S- |
| | MPV-1 | | | (471/502) | Protein/718 |
| | | | | | -741 |
| 20 | CTL- | NTQEVFAQVKQIYKTPPIK | 3 | 91.83% | S- |
| | MPV-1 | | | (461/502) | Protein/777 |
| | | | | | -795 |
| 21 | CTL- | KRSFIEDLLFNKVTLA | 3 | 93.02% | S- |
| | MPV-1 | | | (467/502) | Protein/814 |
| | | | | | -829 |
| 22 | CTL- | PLLTDEMIAQY | 3 | 93.42% | S- |
| | MPV-1 | | | (469/502) | Protein/863 |
| | | | | | -873 |
| 23 | CTL- | LQIPFAMQMAYRENGI | 5 | 93.82% | S- |
| | MPV-1 | | | (471/502) | Protein/894 |
| | | | | | -909 |
| 24 | CTL- | FPQSAPHGVVF | 2 | 93.42% | S- |
| | MPV-1 | | | (469/502) | Protein/105 |
| | | | | | 2-1062 |
| 25 | CTL- | FVSNGTHWFVTQR | 2 | 93.22% | S- |
| | MPV-1 | | | (468/502) | Protein/109 |
| | | | | | 5-1107 |
| 26 | CTL- | YEQYIKWPWYIWLGFIAGLIAIV | 8 | 93.42% | S- |
| | MPV-1 | | | (469/502) | Protein/120 |
| | | | | | 6-1228 |
| 27 | CTL- | NORNAPRITFGGPS | 2 | 96.78% | N- |
| | MPV-2 | | | (482/498) | Protein/8- |
| | | | | | 21 |
| 28 | CTL- | RSKQRRPQGLPNNTASWFTALTQHG | 6 | 99.19% | N- |
| | MPV-2 | K | | (494/498) | Protein/36- |
| | | | | | 61 |
| 29 | CTL- | NSSPDDQIGYYRRATRRIRGGDGKMK | 14 | 97.38% | N- |
| | MPV-2 | DLSPRWYFYYL | | (485/498) | Protein/77- |
| | | | | | 113 |
| 30 | CTL- | LPYGANKDGIIW | 2 | 98.99% | N- |
| | MPV-2 | | | (493/498) | Protein/121 |
| | | | | | -132 |
| 31 | CTL- | KSAAEASKKPRQKRTATKAYNVTQA | 5 | 97.38% | N- |
| | MPV-2 | F | | (485/498) | Protein/249 |
| | | | | | -274 |
| 32 | CTL- | QELIRQGTDY | 2 | 97.18% | N- |
| | MPV-2 | | | (484/498) | Protein/289 |
| | | | | | -298 |
| 33 | CTL- | IAQFAPSASAFFGMSRIGMEVTPSGTW | 12 | 97.18% | N- |
| | MPV-2 | LTY | | (484/498) | Protein/304 |
| | | | | | -333 |
| 34 | CTL- | LLNKHIDAYKTFPPTEPKK | 4 | 96.98% | N- |
| | MPV-2 | | | (483/498) | Protein/352 |
| | | | | | -370 |
| 35 | CTL- | DEWSMATYYLF | 2 | 99.55% | ORF1ab/89 |
| | MPV-2 | | | (451/453) | 8-908/nsp3 |
| 36 | CTL- | ETISLAGSYK | 2 | 100% | ORF1ab/15 |
| | MPV-2 | | | (453/453) | 03- |
| | | | | | 1512/nsp3 |
| 37 | CTL- | TQVVDMSMTY | 2 | 99.77% | ORF1ab/15 |
| | MPV-2 | | | (452/453) | 81- |
| | | | | | 1590/nsp3 |
| 38 | CTL- | AVMYMGTLSYEQF | 3 | 97.79% | ORF1ab/17 |
| | MPV-2 | | | (443/453) | 67- |
| | | | | | 1779/nsp3 |
| 39 | CTL- | LVAEWFLAYILFTRFFYV | 3 | 100% | ORF1ab/23 |
| | MPV-2 | | | (453/453) | 23- |
| | | | | | 2340/nsp3 |
| 40 | CTL- | RMYIFFASFYYVWKSY | 3 | 99.11% | ORF1ab/23 |
| | MPV-2 | | | (449/453) | 82- |
| | | | | | 2397/nsp3 |
| 41 | CTL- | AYVNTFSSTENVPMEK | 3 | 98.89% | ORF1ab/25 |
| | MPV-2 | | | (448/453) | 93- |
| | | | | | 2608/nsp3 |
| 42 | CTL- | CLAYYFMRFRRAF | 3 | 96.02% | ORF1ab/30 |
| | MPV-2 | | | (435/453) | 59- |
| | | | | | 3071/nsp4 |
| 43 | CTL- | MVMFTPLVPFWITIA Y | 4 | 100% | ORF1ab/31 |
| | MPV-2 | | | (453/453) | 29- |
| | | | | | 3144/nsp4 |
| 44 | CTL- | FYWFFSNYLKRRVVF | 2 | 98.67% | ORF1ab/31 |
| | MPV-2 | | | (447/453) | 53- |
| | | | | | 3167/nsp4 |
| 45 | CTL- | LPSLATVAYFNMVY | 2 | 98.01% | ORF1ab/36 |
| | MPV-2 | | | (444/453) | 41- |
| | | | | | 3654/nsp6 |
| 46 | CTL- | LILMTARTVY | 2 | 100% | ORF1ab/36 |
| | MPV-2 | | | (453/453) | 92- |
| | | | | | 3701/nsp6 |
| 47 | CTL- | TACTDDNALAYY | 5 | 99.77% | ORF1ab/41 |
| | MPV-2 | | | (452/453) | 61- |
| | | | | | 4172/nsp9 |
| 48 | CTL- | YTMADLVYAL | 2 | 100% | ORF1ab/45 |
| | MPV-2 | | | (453/453) | 14- |
| | | | | | 4523/nsp12 |

TABLE 2
Identified overlapping epitope cluster based HTL Ag-Patches (antigenic patches
or epitope sequences patches) from the entire proteome of the SARS-CoV-2.
The identified highly

| 49 | CTL- | KLFDRYFKYWDQTY | 2 | 100%(453/ | ORF1ab/46 |
|---|---|---|---|---|---|
| | MPV-2 | | | 453) | 73- |
| | | | | | 4686/nsp12 |
| 50 | CTL- | ARLYYDSMSY | 2 | 99.11% | ORF1ab/49 |
| | MPV-2 | | | (449/453) | 04- |
| | | | | | 4913/nsp12 |
| 51 | CTL- | VDTDFVNEFYAYLRKHFSM | 3 | 100% | ORF1ab/51 |
| | MPV-2 | | | (453/453) | 29- |
| | | | | | 5147/nsp12 |
| 52 | CTL- | FPLCANGQVFGLY | 2 | 99.55% | ORF1ab/54 |
| | MPV-2 | | | (451/453) | 05- |
| | | | | | 5417/nsp13 |
| 53 | CTL- | IPLMYKGLPWNVVR | 2 | 100% | ORF1ab/60 |
| | MPV-2 | | | (453/453) | 75- |
| | | | | | 6088/nsp14 |
| 54 | CTL- | YVMHANYIFW | 2 | 98.89% | ORF1ab/70 |
| | MPV-2 | | | (448/453) | 20- |
| | | | | | 7029/nsp16 |
| 55 | CTL- | SEETGTLIVNSVLLFLAFVVFLLVTLAI | 20 | 99.17% | E- |
| | MPV-3 | LTALRLCAY | | (478/482) | Protein/6- |
| | | | | | 42 |
| 56 | CTL- | VSLVKPSFYVYSRVKNLNSSR | 5 | 99.58% | E- |
| | MPV-3 | | | (480/482) | Protein/49- |
| | | | | | 69 |
| 57 | CTL- | FMRIFTIGTVTLK | 5 | 99.37% | ORF3a/4- |
| | MPV-3 | | | (478/481) | 16 |
| 58 | CTL- | TIPIQASLPFGWLIVGVALLA VFQSAS | 14 | 77.33% | ORF3a/34- |
| | MPV-3 | KIITLKKRW | | (372/481) | 69 |
| 59 | CTL- | HFVCNLLLLFVTV | 5 | 97.50% | ORF3a/78- |
| | MPV-3 | | | (469/481) | 90 |
| 60 | CTL- | LLVAAGLEAPFLYLYALVYFLQSINFV | 27 | 95.42% | ORF3a/95- |
| | MPV-3 | RIIMRLWLCWKCR | | (459/481) | 134 |
| 61 | CTL- | YFLCWHTNCYDY | 3 | 97.08% | ORF3a/145 |
| | MPV-3 | | | (467/481) | -156 |
| 62 | CTL- | TTSPISEHDYQIGGY | 2 | 97.92% | ORF3a/175 |
| | MPV-3 | | | (471/481) | -189 |
| 63 | CTL- | YYQLYSTQLSTDTGVEHVTFFIYNKI | 6 | 99.16% | ORF3a/211 |
| | MPV-3 | | | (477/481) | -236 |
| 64 | CTL- | FHLVDFQVTIAEILLIIMRTFKVSIWNL | 12 | 98.33% | ORF6/2-33 |
| | MPV-3 | DYII | | (473/481) | |
| 65 | CTL- | MKIILFLALITLATCELYHY | 7 | 99.58% | ORF7a/1- |
| | MPV-3 | | | (478/480) | 20 |
| 66 | CTL- | LLKEPCSSGTYEGNSPFHPLADNKFAL | 18 | 98.75% | ORF7a/30- |
| | MPV-3 | TCFSTQFAFACPDGVKHVYQLRARSV | | (474/480) | 89 |
| | | SPKLFIR | | | |
| 67 | CTL- | EVQELYSPIFLIVAAIVFITLCFTLKRK | 11 | 97.70% | ORF7a/92- |
| | MPV-3 | | | (469/480) | 119 |
| 68 | CTL- | MIELSLIDFYLCFLAFLLFLVLIMLIIFW | 24 | 97.88% | ORF7b/1- |
| | MPV-3 | FSLEL | | (231/236) | 34 |
| 69 | CTL- | MKFLVFLGIITTVAAF | 9 | 98.95% | ORF8/1-16 |
| | MPV-3 | | | (475/480) | |
| 70 | CTL- | YVVDDPCPIHFYSKWYIRVGARKSAP | 12 | 98.95% | ORF8/31- |
| | MPV-3 | LIELC | | (475/480) | 61 |
| 71 | CTL- | IQYIDIGNYTVSCLPFTINCQEPKLGSL | 19 | 51.25% | ORF8/71- |
| | MPV-3 | VVRCSFYEDFLEYHDVRVVL | | (246/480) | 118 |
| 72 | CTL- | IQYIDIGNYTVSCSPFTINCQEPKLGSL | 19 | 47.50% | ORF8/71- |
| | MPV-3 | VVRCSFYEDFLEYHDVRVVL | | (228/480) | 118 |
| 73 | CTL- | MGYINVFAFPFTIYSLLLCR | 15 | 99.37% | ORF10/1- |
| | MPV-3 | | | (476/479) | 20 |
| | | | # of | | Source |
| Seq. | | | epitopes | | SARS- |
| ID | HTL- | CTL epitope cluster based | in | | CoV-2 |
| No. | MPVs | patches | cluster | Conserved | protein |
| 74 | HTL-MPV- | VNSVLLFLAFVVFLLVTLAI | 8 | 99.58%(480 | E- |
| | 1 | LT | | /482) | Protein/14- |
| | | | | | 35 |
| 75 | HTL-MPV- | IKLIFLWLLWPVTLAC | 2 | 98.11%(468 | m- |
| | 1 | | | /477) | protein/49- |
| | | | | | 64 |
| 76 | HTL-MPV- | VGLMWLSYFIASFRLFAR | 4 | 97.27%(464 | m- |
| | 1 | | | /477) | protein/88- |
| | | | | | 105 |
| 77 | HTL-MPV- | SRTLSYYKLGASQRVAGDS | 5 | 98.53%(470 | m- |
| | 1 | | | /477) | protein/173 |
| | | | | | -191 |
| 78 | HTL-MPV- | SKTQSLLIVNNATNVVIKVC | 7 | 93.22%(468 | S- |
| | 1 | E | | /502) | protein/112 |
| | | | | | -132 |
| 79 | HTL-MPV- | REFVFKNIDGYFKIYSK | 3 | 91.83%(461 | S- |
| | 1 | | | /502) | protein/190 |
| | | | | | -206 |
| 80 | HTL-MPV- | GINITRFQTLLALHRSYLTPG | 9 | 92.82%(466 | S- |
| | 1 | DSSSGWTAGAAAYYVGY | | /502) | protein/232 |
| | | | | | -269 |
| 81 | HTL-MPV- | QPTESIVRFPNITNLCPFG | 5 | 93.42%(469 | S- |
| | 1 | | | /502) | protein/321 |
| | | | | | -339 |
| 82 | HTL-MPV- | ADYSVLYNSASFSTFKC | 3 | 93.82%(471 | S- |
| | 1 | | | /502) | protein/363 |
| | | | | | -379 |
| 83 | HTL-MPV- | YADSFVIRGDEVRQIAPGQ | 5 | 93.62%(470 | S- |
| | 1 | | | /502) | protein/396 |
| | | | | | -414 |
| 84 | HTL-MPV- | VVVLSFELLHAPATVCGPK | 5 | 92.23%(463 | S- |
| | 1 | | | /502) | protein/510 |
| | | | | | -528 |
| 85 | HTL-MPV- | IAIPTNFTISVTTEILPVS | 5 | 93.82%(471 | S- |
| | 1 | | | /502) | protein/712 |
| | | | | | -730 |
| 86 | HTL-MPV- | ECSNLLLQYGSFCTQLNR | 4 | 93.82%(471 | S- |
| | 1 | | | /502) | protein/748 |
| | | | | | -765 |
| 87 | HTL-MPV- | QQLIRAAEIRASANLA | 2 | 93.22%(468 | S- |
| | 1 | | | /502) | protein/101 |
| | | | | | 0-1025 |
| 88 | HTL-MPV- | REGVFVSNGTHWFVTQ | 2 | 93.22%(468 | S- |
| | 1 | | | /502) | protein/109 |
| | | | | | 1-1106 |
| 89 | HTL-MPV- | PWYIWLGFIAGLIAIVMVTI | 9 | 93.42%(469 | S- |
| | 1 | MLCC | | /502) | protein/121 |
| | | | | | 3-1236 |
| 90 | HTL-MPV- | DDQIGYYRRATRRIRGGDG | 5 | 98.79%(492 | N- |
| | 1 | | | /498) | Protein/81- |
| | | | | | 99 |
| 91 | HTL-MPV- | GTRNPANNAAIVLQLPQGTT | 7 | 97.38%(485 | N- |
| | 1 | L | | /498) | Protein/147 |
| | | | | | -167 |
| 92 | HTL-MPV- | WPQIAQFAPSASAFFGMSR | 5 | 97.38%(485 | N- |
| | 1 | | | /498) | Protein/301 |
| | | | | | -319 |
| 93 | HTL-MPV- | TPSGTWLTYTGAIKLDDKD | 5 | 97.18%(484 | N- |
| | 1 | | | /498) | Protein/325 |
| | | | | | -343 |
| 94 | HTL-MPV- | SDFVRATATIPIQASLP | 3 | 99.37%(478 | 3a/26-42 |
| | 1 | | | /481) | |
| 95 | HTL-MPV- | APFLYLYALVYFLQSINFV | 5 | 96.46%(464 | 3a/103-121 |
| | 1 | | | /481) | |
| 96 | HTL-MPV- | DTGVEHVTFFIYNKIVDEPE | 7 | 99.37%(478 | 3a/222-244 |
| | 1 | EHV | | /481) | |
| 97 | HTL-MPV- | AIILASFSASTSAFVET | 3 | 100%(453/ | 1 ab/474- |
| | 2 | | | 453) | 490/nsp2 |
| 98 | HTL-MPV- | YAFASEAARVVRSIFS | 2 | 99.55%(451 | 1ab/534- |
| | 2 | | | /453) | 549/nsp2 |
| 99 | HTL-MPV- | TLEETKFLTENLLLYIDIN | 5 | 99.77%(452 | 1ab/1242- |
| | 2 | | | /453) | 1260/nsp3 |
| 100 | HTL-MPV- | VQQESPFVMMSAPPAQYEL | 5 | 99.77%(452 | 1ab/1798- |
| | 2 | | | /453) | 1816/nsp3 |
| 101 | HTL-MPV- | LLQLCTFTRSTNSRIKASM | 5 | 98.23%(445 | 1 ab/2176- |
| | 2 | | | /453) | 2194/nsp3 |
| 102 | HTL-MPV- | SKLINIIIWFLLLSVCL | 3 | 97.79%(443 | 1ab/2224- |
| | 2 | | | /453) | 2240/nsp3 |
| 103 | HTL-MPV- | EWFLAYILFTRFFYVLGLAA | 17 | 100%(453/ | 1ab/2326- |
| | 2 | IMQLFFSYFA VHFISNSWLM | | 453) | 2375/nsp3 |
| | | WLIINLVQMA | | | |
| 104 | HTL-MPV- | VRMYIFFASFYYVWKSYV | 4 | 98.45%(446 | 1ab/2381- |
| | 2 | | | /453) | 2398/nsp3 |
| 105 | HTL-MPV- | WLKQLIKVTLVFLFVAAIFY | 9 | 99.77%(452 | 1 ab/2769- |
| | 2 | LITPVH | | /453) | 2794/nsp4 |
| 106 | HTL-MPV- | FSAVGNICYTPSKLIEYT | 4 | 99.55%(451 | 1ab/2889- |
| | 2 | | | /453) | 2906/nsp4 |
| 107 | HTL-MPV- | DISASIVAGGIVAIVVTCL | 5 | 100%(453/ | lab/3042- |
| | 2 | | | 453) | 3060/nsp4 |
| 108 | HTL-MPV- | FTPLVPFWITIA YIICIST | 5 | 100%(453/ | 1ab/3132- |
| | 2 | | | 453) | 3150/nsp4 |
| 109 | HTL-MPV- | VQSTQWSLFFFLYENAFLP | 5 | 92.27%(418 | 1ab/3595- |
| | 2 | | | /453) | 3613/nsp6 |
| 110 | HTL-MPV- | QAIASEFSSLPSYAAFAT | 4 | 99.33%(450 | 1ab/3942- |
| | 2 | | | /453) | 3959/nsp8 |
| 111 | HTL-MPV- | TQMNLKYAISAKNRART | 3 | 99.77%(452 | 1ab/4932- |
| | 2 | | | /453) | 4948/nsp12 |
| 112 | HTL-MPV- | RAMPNMLRIMASLVLARKH | 5 | 100%(453/ | 1ab/5016- |
| | 2 | | | 453) | 5034/nsp12 |
| 113 | HTL-MPV- | FVNEFYAYLRKHFSMMIL | 4 | 100%(453/ | 1ab/5133- |
| | 2 | | | 453) | 5150/nsp12 |
| 114 | HTL-MPV- | PEVKILNNLGVDIAAN | 2 | 99.77%(452 | 1ab/6519- |
| | 2 | | | /453) | 6534/nsp15 |
| 115 | HTL-MPV- | SQMEIDFLELAMDEFIERY | 5 | 99.11%(449 | 1ab/6659- |
| | 2 | | | /453) | 6677/nsp15 |
| 116 | HTL-MPV- | FAWWTAFVTNVNASSS | 2 | 98.89%(448 | 1ab/6985- |
| | 2 | | | /453) | 7000/nsp16 |
| 117 | HTL-MPV- | HANYIFWRNTNPIQLSSY | 4 | 98.89%(448 | lab/7023- |
| | 2 | | | /453) | 7040/nsp16 |
| 118 | HTL-MPV- | EILLIIMRTFKVSIWNLDYIIN | 6 | 99.16%(477 | orf6/13-38 |
| | 2 | LIIK | | /481) | |
| 119 | HTL-MPV- | MKIILFLALITLATCELY | 4 | 99.37%(478 | orf7a/1-18 |
| | 2 | | | /481) | |
| 120 | HTL-MPV- | LSLIDFYLCFLAFLLFLVLIM | 14 | 97.88%(231 | 7b/4-34 |
| | 2 | LIIFWFSLEL | | /236) | |
| 121 | HTL-MPV- | CTQHQPYVVDDPCPIHFYS | 5 | 99.16%(476 | orf8/25-43 |
| | 2 | | | /480) | |
| 122 | HTL-MPV- | YINVFAFPFTIYSLLLCRM | 5 | 99.37%(476 | orf10/3-21 |
| | 2 | | | /479) | |

TABLE 3
Five vaccine constructs as designed by using the CTL and HTL patches from the
proteome of SARS-CoV-2 as identified are shown, called CTL-MPV-1, CTL-MPV-2, CTL-
MPV-3, HTL-MPV-1, and HTL-MPV-2, respectively. Vaccines as designed consist of two
adjuvant sequences, GIGDPVTCLKSGAICHPVFCPRRYKQIGTCGLPGTKCCKKP (SEQ
ID NO: 128) and GIINTLQKYYCRVRGGRCAVLSCLPKEEQIGKCSTRGRKCCRRKK
(SEQ ID NO: 129); linkers: GGGGS (SEQ ID NO: 130) and EAAAK (SEQ ID NO: 131); and
6X histidine tags: HHHHHH (SEQ ID NO: 132) as well as the CTL and HTL Ag-Patches
(antigenic patches or epitope sequences patches) from Tables 1 or 2, as above.

| Seq. | | |
|---|---|---|
| ID | | |
| No. | Vaccine | Vaccine construct |
| 123 | CTL-MPV-1 | GIGDPVTCLKSGAICHPVFCPRRYKQIGTCGLPGTKCCK |
| | construct | KPEAAAKGTITVEELKKLLEQWNLVIGFLFLTWICLLQF |
| | (Comprising of | AYANRNRFLYIIKLIFLWLLWPVTLACFVLAAVGGGGSR |
| | identified CTL | INWITGGIAIAMACLVGLMWLSYFIASFRLFARTRSMWS |
| | Ag-Patches from | FNGGGGSKEITVATSRTLSYYKLGASQRVAGDSGFAAYS |
| | Membrane and | RYRIGNYKLGGGGSTTRTQLPPAYTNSFTRGVYYPDKVF |
| | Spike protein of | RSSVGGGGSSTQDLFLPFFSNVTWFGGGGSVLPFNDGVY |
| | SARS-CoV-2): | FASTEKSNIIRGWIFGGGGSGVYYHKNNKSWMESEFRV |
| | | YSSANNCTFEYVSQPFLGGGGSKQGNFKNLREFVFKNID |
| | | GYFKIYSKHTPIGGGGSEPLVDLPIGINITRFQTLLAGGGG |
| | | STPGDSSSGWTAGAAAYYVGYLQPRTFLLKGGGGSSET |
| | | KCTLKSFTVEKGIYQTSNFRGGGGSFPNITNLCPFGEVEN |
| | | ATRFASVGGGGSRISNCVADYSVLYNSASFSTFKCYGGG |
| | | GSNVYADSFVIRGDEVRGGGGSSKVGGNYNYLYRLFRK |
| | | SNLKPFERGGGGSNTSNQVAVLYQDVNCTEVGGGGSRV |
| | | YSTGSNVFGGGGSSPRRARSVASQSIIAYGGGGSFTISVT |
| | | TEILPVSMTKTSVDCTMYGGGGSNTQEVFAQVKQIYKTP |
| | | PIKGGGGSKRSFIEDLLFNKVTLAGGGGSPLLTDEMIAQY |
| | | GGGGSLQIPFAMQMAYRENGIGGGGSFPQSAPHGVVFG |
| | | GGGSFVSNGTHWFVTQRGGGGSYEQYIKWPWYIWLGFI |
| | | AGLIAIVEAAAKGIINTLQKYYCRVRGGRCAVLSCLPKE |
| | | EQIGKCSTRGRKCCRRKKHHHHHH |
| 124 | CTL-MPV-2 | GIGDPVTCLKSGAICHPVFCPRRYKQIGTCGLPGTKCCK |
| | construct | KPEAAAKNQRNAPRITFGGPSGGGGSRSKQRRPQGLPNN |
| | (Comprising of | TASWFTALTQHGKGGGGSNSSPDDQIGYYRRATRRIRG |
| | identified CTL | GDGKMKDLSPRWYFYYLGGGGSLPYGANKDGIIWGGG |
| | Ag-Patches from | GSKSAAEASKKPRQKRTATKAYNVTQAFGGGGSQELIR |
| | Nucleocapsid | QGTDYGGGGSIAQFAPSASAFFGMSRIGMEVTPSGTWLT |
| | protein and | YGGGGSLLNKHIDAYKTFPPTEPKKGGGGSDEWSMATY |
| | ORF1ab proteins | YLFGGGGSETISLAGSYKGGGGSTQVVDMSMTYGGGGS |
| | of SARS-CoV-2): | AVMYMGTLSYEQFGGGGSLVAEWFLAYILFTRFFYVGG |
| | | GGSRMYIFFASFYYVWKSYGGGGSAYVNTFSSTFNVPM |
| | | EKGGGGSCLAYYFMRFRRAFGGGGSMVMFTPLVPFWIT |
| | | IAYGGGGSFYWFFSNYLKRRVVFGGGGSLPSLATVAYF |
| | | NMVYGGGGSLILMTARTVYGGGGSTACTDDNALAYYG |
| | | GGGSYTMADLVYALGGGGSKLFDRYFKYWDQTYGGG |
| | | GSARLYYDSMSYGGGGSVDTDFVNEFYAYLRKHFSMG |
| | | GGGSFPLCANGQVFGLYGGGGSIPLMYKGLPWNVVRG |
| | | GGGSYVMHANYIFWEAAAKGIINTLQKYYCRVRGGRC |
| | | AVLSCLPKEEQIGKCSTRGRKCCRRKKHHHHHH |
| 125 | CTL-MPV-3 | GIGDPVTCLKSGAICHPVFCPRRYKQIGTCGLPGTKCCK |
| | construct | KPEAAAKSEETGTLIVNSVLLFLAFVVFLLVTLAILTALRLCA |
| | (Comprising of | YGGGGSVSLVKPSFYVYSRVKNLNSSRGGGGSFMRIFTIGTV |
| | identified CTL | TLKGGGGSTIPIQASLPFGWLIVGVALLAVFQSASKIITLK |
| | Ag-Patches from | KRWGGGGSHFVCNLLLLFVTVGGGGSLLVAAGLEAPFL |
| | Envelope protein, | YLYALVYFLQSINFVRIIMRLWLCWKCRGGGGSYFLCW |
| | ORF3a protein, | HTNCYDYGGGGSTTSPISEHDYQIGGYGGGGSYYQLYST |
| | ORF6 protein, | QLSTDTGVEHVTFFIYNKIGGGGSFHLVDFQVTIAEILLII |
| | ORF7a protein, | MRTFKVSIWNLDYIIGGGGSMKIILFLALITLATCELYHY |
| | ORF7b protein, | GGGGSLLKEPCSSGTYEGNSPFHPLADNKFALTCFSTQF |
| | ORF8 protein, | AFACPDGVKHVYQLRARSVSPKLFIRGGGGSEVQELYSP |
| | and ORF10 | IFLIVAAIVFITLCFTLKRKGGGGSMIELSLIDFYLCFLAFL |
| | protein of SARS- | LFLVLIMLIIFWFSLELGGGGSMKFLVFLGIITTVAAFGGG |
| | CoV-2): | GSYVVDDPCPIHFYSKWYIRVGARKSAPLIELCGGGGSIQ |
| | | YIDIGNYTVSCLPFTINCQEPKLGSLVVRCSFYEDFLEYH |
| | | DVRVVLGGGGSMGYINVFAFPFTIYSLLLCREAAAKGIIN |
| | | TLQKYYCRVRGGRCAVLSCLPKEEQIGKCSTRGRKCCR |
| | | RKKHHHHHH |
| 126 | HTL-MPV-1 | GIGDPVTCLKSGAICHPVFCPRRYKQIGTCGLPGTKCCK |
| | construct | KPEAAAKVNSVLLFLAFVVFLLVTLAILTGGGGSIKLIFL |
| | (Comprising of | WLLWPVTLACGGGGSVGLMWLSYFIASFRLFARGGGGS |
| | identified CTL | SRTLSYYKLGASQRVAGDSGGGGSSKTQSLLIVNNATN |
| | Ag-Patches from | VVIKVCEGGGGSREFVFKNIDGYFKIYSKGGGGSGINITR |
| | Envelope protein, | FQTLLALHRSYLTPGDSSSGWTAGAAAYYVGYGGGGSQ |
| | Membrane | PTESIVRFPNITNLCPFGGGGGSADYSVLYNSASFSTFKC |
| | protein, Spike | GGGGSYADSFVIRGDEVRQIAPGQGGGGSVVVLSFELLH |
| | protein, | APATVCGPKGGGGSIAIPTNFTISVTTEILPVSGGGGSQQL |
| | Nucleocapsid | IRAAEIRASANLAGGGGSREGVFVSNGTHWFVTQGGGG |
| | protein, ORF3a | SPWYIWLGFIAGLIAIVMVTIMLCCGGGGSDDQIGYYRR |
| | protein of SARS- | ATRRIRGGDGGGGGSGTRNPANNAAIVLQLPQGTTLGG |
| | CoV-2): | GGSWPQIAQFAPSASAFFGMSRGGGGSTPSGTWLTYTG |
| | | AIKLDDKDGGGGSSDFVRATATIPIQASLPGGGGSAPFLY |
| | | LYALVYFLQSINFVGGGGSDTGVEHVTFFIYNKIVDEPEE |
| | | HVEAAAKGIINTLQKYYCRVRGGRCAVLSCLPKEEQIGK |
| | | CSTRGRKCCRRKKHHHHHH |
| 127 | HTL-MPV-2 | GIGDPVTCLKSGAICHPVFCPRRYKQIGTCGLPGTKCCK |
| | construct | KPEAAAKAIILASFSASTSAFVETGGGGSYAFASEAARV |
| | (Comprising of | VRSIFSGGGGSTLEETKELTENLLLYIDINGGGGSVQQES |
| | identified CTL | PFVMMSAPPAQYELGGGGSLLQLCTFTRSTNSRIKASMG |
| | Ag-Patches from | GGGSSKLINIIIWFLLLSVCLGGGGSEWFLAYILFTRFFYV |
| | ORF1ab protein, | LGLAAIMQLFFSYFAVHFISNSWLMWLIINLVQMAGGG |
| | ORF6 protein, | GSVRMYIFFASFYYVWKSYVGGGGSWLKQLIKVTLVFL |
| | ORF7a protein, | FVAAIFYLITPVHGGGGSFSAVGNICYTPSKLIEYTGGGG |
| | ORF7b protein, | SDISASIVAGGIVAIVVTCLGGGGSFTPLVPFWITIAYIICIS |
| | ORF8 protein, | TGGGGSVQSTQWSLFFFLYENAFLPGGGGSQAIASEFSSL |
| | ORF10 protein) | PSYAAFATGGGGSTQMNLKYAISAKNRARTGGGGSRA |
| | of SARS-CoV-2): | MPNMLRIMASLVLARKHGGGGSFVNEFYAYLRKHFSM |
| | | MILGGGGSPEVKILNNLGVDIAANGGGGSSQMEIDFLEL |
| | | AMDEFIERYGGGGSFAWWTAFVTNVNASSSGGGGSHA |
| | | NYIFWRNTNPIQLSSYGGGGSEILLIIMRTFKVSIWNLDYI |
| | | INLIIKGGGGSMKIILFLALITLATCELYGGGGSLSLIDFYL |
| | | CFLAFLLFLVLIMLIIFWFSLELGGGGSCTQHQPYVVDDP |
| | | CPIHFYSGGGGSYINVFAFPFTIYSLLLCRMEAAAKGIINT |
| | | LQKYYCRVRGGRCAVLSCLPKEEQIGKCSTRGRKCCRR |
| | | KKHHHHHH |

Of the clusters observed, one cluster for each protein (M, E, and N protein) is shown in FIG. 3 as an example.

Step 2 thus provided one Ag-Patch from each of the overlapping epitope clusters for SARS-Cov-2 proteins as follows (FIG. 3):
a) Envelope protein Ag-Patch

(SEQ ID NO: 3)
KEITVATSRTLSYYKLGASQRVAGDSGFAAYSRYRIGNYKL,

identified by clustering of 21 overlapping epitopes.
b) Membrane protein Ag-Patch

(SEQ ID NO: 55)
SEETGTLIVNSVLLFLAFVVFLLVTLAILTALRLCAY,

identified by clustering of 20 overlapping epitopes.
c) Nucleocapsid protein Ag-Patch

(SEQ ID NO: 29)
NSSPDDQIGYYRRATRRIRGGDGKMKDLSPRWYFYYL,

identified by clustering of 14 overlapping epitopes.

### Example 2

Step 3: Design of a Multi-Patch Vaccine using the Ag-Patches (antigenic patches or epitope sequences patches) as identified in Example 1.

The Ag-Patches identified in Example 1 from the three proteins of SARS-CoV-2 were used here to design Multi-Patch Vaccine to represent an example for a Multi-Patch Vaccine design from the Ag-Patches identified by the “Overlapping-Epitope-Clusters-To-Patches” method (FIG. 3, Table 1 to 3).

In the above-identified embodiment, the Ag-Patches (antigenic patches or epitope sequences patches) as identified were fused together by short peptide linkers GGGGS (SEQ ID NO: 130), as shown in FIG. 3 (Table 3). In this example of the Multi-Patch Vaccine, the Ag-Patches identified by the “Overlapping-Epitope-Clusters-To-Patches” method were utilized. In addition to this MPV design, the MPV may also be fused with adjuvant proteins like, e.g., truncated (residues 10-153) Onchocerca volvulus activation-associated secreted protein-1 fused to the N and/or C terminal of the MPV, to enhance the immune response.

### REFERENCES AS CITED

Grifoni, A., Sidney, J., Zhang, Y., Scheuermann, R. H., Peters, B. and Sette, A., 2020. A sequence homology and bioinformatic approach can predict candidate targets for immune responses to SARS-CoV-2. Cell host & microbe, 27(4), pp. 671-680.
Srivastava, S., Verma, S., Kamthania, M., Kaur, R., Badyal, R. K., Saxena, A. K., Shin, H. J., Kolbe, M. and Pandey, K. C., 2020. Structural basis for designing multiepitope vaccines against COVID-19 infection: In silico vaccine design and validation. JMIR bioinformatics and biotechnology, 1(1), p.e19371.
Srivastava, S., Verma, S., Kamthania, M., Agarwal, D., Saxena, A. K., Kolbe, M., Singh, S., Kotnis, A., Rathi, B., Nayar, S. A. and Shin, H. J., 2020. Computationally validated SARS-CoV-2 CTL and HTL Multi-Patch vaccines, designed by reverse epitomics approach, show potential to cover large ethnically distributed human population worldwide. Journal of Biomolecular Structure and Dynamics, pp. 1-20.

1. A method for producing an immunogenic composition of peptide sequences from at least one proteome of interest, comprising the steps of:
a) providing multiple epitope sequences from said at least one proteome of interest,
b) aligning the multiple epitope sequences of a) in order to generate at least one immunogenic peptide sequence comprising an antigenic patch (Ag-Patch or epitope sequences patches), consisting of at least two overlapping epitope sequences from a region of a polypeptide from said at least one proteome of interest, and
c) combining said Ag-Patch (antigenic patches or epitope sequences patches) into an immunogenic polypeptide construct, the construct comprising at least one Ag-Patch, at least one linker peptide sequence, and at least one adjuvant peptide sequence, and thereby producing an immunogenic composition of peptide sequences from at least one proteome of interest.

2. The method according to claim 1, wherein said Ag-Patches (antigenic patches or epitope sequences patches) are identical to at least 90% on the amino acid level between different related proteomes of interest.

3. The method according to claim 1, wherein said proteome of interest is derived from bacteria, parasites or fungi; cancerous cells; plant cells; viruses; cells recombinantly expressing proteins; or infected cells.

4. The method according to claim 1, wherein said proteome of interest comprises preselected polypeptides selected from the group of membrane- or membrane-associated polypeptides, extracellular polypeptides, non-cytosolic polypeptides, envelope polypeptides, and polypeptides that are accessible to binding by antibodies and/or T-cells.

5. The method according to claim 1, wherein said epitope is an antibody binding epitope or an epitope for an antigen binding fragment thereof, or a T-cell receptor binding epitope or an epitope for an antigen binding fragment thereof.

6. The method according to claim 1, wherein said epitopes are from two, three, four or more proteomes of interest from different strains or members of the same species or genus.

7. The method according to claim 1, wherein said Ag-Patch (antigenic patches or epitope sequences patches) comprises between 2 and 20 epitopes from a region of a polypeptide from said at least one proteome of interest.

8. The method according to claim 1, wherein said immunogenic composition of peptide sequences comprises one or more Ag-Patches (antigenic patches or epitope sequences patches).

9. The method according to claim 1, wherein said at least one linker peptide sequence is GGGGS (SEQ ID NO: 130) or EAAAK (SEQ ID NO: 131).

10. The method according to claim 1, wherein said at least one adjuvant peptide sequence is selected from, human P defensin 2 (GIGDPVTCLKSGAICHPVFCPRRYKQIGTCGLPGTKCCKKP) (SEQ ID NO: 128) and human P defensin 3 (GIINTLQKYYCRVRGGRCAVLSCLPKEEQIGK CSTRG-RKCCRRKK) (SEQ ID NO: 129), and is positioned at the N- and/or C-terminus of the immunogenic peptide sequences construct.

11. The method according to claim 1, wherein said immunogenic composition furthermore comprises at least one tag peptide sequence.

12. The method according to claim 1, further comprising expressing at least one nucleic acid sequence encoding said immunogenic composition of peptide sequences in a suitable host cell.

13. An immunogenic composition of peptide sequences, produced according to claim 1.

14. The immunogenic composition of peptide sequences according to claim 13 in form of a pharmaceutical composition, a vaccine or a diagnostic kit.

15. The immunogenic composition of peptide sequences according to claim 13, wherein the at least one Ag-Patch (antigenic patches or epitope sequences patches) is further selected based on the ethnic diversity of HLA alleles in a human population.

16. The immunogenic composition of peptide sequences according to claim 13, selected from the group of polypeptides consisting of SEQ ID NO: 123 to SEQ ID NO: 127.

17. A therapeutic or diagnostic kit, comprising the immunogenic composition of peptide sequences according to claim 13, together with suitable auxiliary agents, packaging, and/or instructions for use.

18. A method for detecting a proteome of interest, wherein said proteome of interest is derived from bacteria, parasites or fungi; cancerous cells; plant cells; viruses; cells recombinantly expressing proteins; or infected cells, wherein said method comprises the use of an immunogenic composition of peptide sequences, produced according to claim 1.

19. A method for identifying and/or producing a set of antibodies, T-cell receptors and/or T-cells or B-cells that are specific for a proteome of interest, wherein said proteome of interest is derived from bacteria, parasites or fungi; cancerous cells; plant cells; viruses; or infected cells, wherein said method comprises the use of an immunogenic composition of peptide sequences, produced according to claim 1.

20. (canceled)

21. A method for preventing and/or treating a condition or disease selected from the group consisting of an infection by a bacteria, parasite, fungi or pathogenic viruses, and cancer, in a subject in need thereof, comprising administering to said subject an effective amount of the immunogenic composition of peptide sequences according to claim 13.


SEQUENCE LISTING (200 sequences)

SEQ ID NO: 1 - protein, 65 aa, Corona virus SARS-CoV-2
GTITVEELKKLLEQWNLVIGFLFLTWICLLQFAYANRNRFLYIIKLIFLWLLWPVTLACFVLAAV

SEQ ID NO: 2 - protein, 42 aa, Corona virus SARS-CoV-2
RINWITGGIAIAMACLVGLMWLSYFIASFRLFARTRSMWSFN

SEQ ID NO: 3 - protein, 41 aa, Corona virus SARS-CoV-2
KEITVATSRTLSYYKLGASQRVAGDSGFAAYSRYRIGNYKL

SEQ ID NO: 4 - protein, 29 aa, Corona virus SARS-CoV-2
TTRTQLPPAYTNSFTRGVYYPDKVFRSSV

SEQ ID NO: 5 - protein, 16 aa, Corona virus SARS-CoV-2
STQDLFLPFFSNVTWF

SEQ ID NO: 6 - protein, 24 aa, Corona virus SARS-CoV-2
VLPFNDGVYFASTEKSNIIRGWIF

SEQ ID NO: 7 - protein, 35 aa, Corona virus SARS-CoV-2
GVYYHKNNKSWMESEFRVYSSANNCTFEYVSQPFL

SEQ ID NO: 8 - protein, 29 aa, Corona virus SARS-CoV-2
KQGNFKNLREFVFKNIDGYFKIYSKHTPI

SEQ ID NO: 9 - protein, 20 aa, Corona virus SARS-CoV-2
EPLVDLPIGINITRFQTLLA

SEQ ID NO: 10 - protein, 29 aa, Corona virus SARS-CoV-2
TPGDSSSGWTAGAAAYYVGYLQPRTFLLK

SEQ ID NO: 11 - protein, 23 aa, Corona virus SARS-CoV-2
SETKCTLKSFTVEKGIYQTSNFR

SEQ ID NO: 12 - protein, 22 aa, Corona virus SARS-CoV-2
FPNITNLCPFGEVFNATRFASV

SEQ ID NO: 13 - protein, 24 aa, Corona virus SARS-CoV-2
RISNCVADYSVLYNSASFSTFKCY

SEQ ID NO: 14 - protein, 15 aa, Corona virus SARS-CoV-2
NVYADSFVIRGDEVR

SEQ ID NO: 15 - protein, 24 aa, Corona virus SARS-CoV-2
SKVGGNYNYLYRLFRKSNLKPFER

SEQ ID NO: 16 - protein, 18 aa, Corona virus SARS-CoV-2
NTSNQVAVLYQDVNCTEV

SEQ ID NO: 17 - protein, 10 aa, Corona virus SARS-CoV-2
RVYSTGSNVF

SEQ ID NO: 18 - protein, 16 aa, Corona virus SARS-CoV-2
SPRRARSVASQSIIAY

SEQ ID NO: 19 - protein, 24 aa, Corona virus SARS-CoV-2
FTISVTTEILPVSMTKTSVDCTMY

SEQ ID NO: 20 - protein, 19 aa, Corona virus SARS-CoV-2
NTQEVFAQVKQIYKTPPIK

SEQ ID NO: 21 - protein, 16 aa, Corona virus SARS-CoV-2
KRSFIEDLLFNKVTLA

SEQ ID NO: 22 - protein, 11 aa, Corona virus SARS-CoV-2
PLLTDEMIAQY

SEQ ID NO: 23 - protein, 16 aa, Corona virus SARS-CoV-2
LQIPFAMQMAYRFNGI

SEQ ID NO: 24 - protein, 11 aa, Corona virus SARS-CoV-2
FPQSAPHGVVF

SEQ ID NO: 25 - protein, 13 aa, Corona virus SARS-CoV-2
FVSNGTHWFVTQR

SEQ ID NO: 26 - protein, 23 aa, Corona virus SARS-CoV-2
YEQYIKWPWYIWLGFIAGLIAIV

SEQ ID NO: 27 - protein, 14 aa, Corona virus SARS-CoV-2
NQRNAPRITFGGPS

SEQ ID NO: 28 - protein, 26 aa, Corona virus SARS-CoV-2
RSKQRRPQGLPNNTASWFTALTQHGK

SEQ ID NO: 29 - protein, 37 aa, Corona virus SARS-CoV-2
NSSPDDQIGYYRRATRRIRGGDGKMKDLSPRWYFYYL

SEQ ID NO: 30 - protein, 12 aa, Corona virus SARS-CoV-2
LPYGANKDGIIW

SEQ ID NO: 31 - protein, 26 aa, Corona virus SARS-CoV-2
KSAAEASKKPRQKRTATKAYNVTQAF

SEQ ID NO: 32 - protein, 10 aa, Corona virus SARS-CoV-2
QELIRQGTDY

SEQ ID NO: 33 - protein, 30 aa, Corona virus SARS-CoV-2
IAQFAPSASAFFGMSRIGMEVTPSGTWLTY

SEQ ID NO: 34 - protein, 19 aa, Corona virus SARS-CoV-2
LLNKHIDAYKTFPPTEPKK

SEQ ID NO: 35 - protein, 11 aa, Corona virus SARS-CoV-2
DEWSMATYYLF

SEQ ID NO: 36 - protein, 10 aa, Corona virus SARS-CoV-2
ETISLAGSYK

SEQ ID NO: 37 - protein, 10 aa, Corona virus SARS-CoV-2
TQVVDMSMTY

SEQ ID NO: 38 - protein, 13 aa, Corona virus SARS-CoV-2
AVMYMGTLSYEQF

SEQ ID NO: 39 - protein, 18 aa, Corona virus SARS-CoV-2
LVAEWFLAYILFTRFFYV

SEQ ID NO: 40 - protein, 16 aa, Corona virus SARS-CoV-2
RMYIFFASFYYVWKSY

SEQ ID NO: 41 - protein, 16 aa, Corona virus SARS-CoV-2
AYVNTFSSTFNVPMEK

SEQ ID NO: 42 - protein, 13 aa, Corona virus SARS-CoV-2
CLAYYFMRFRRAF

SEQ ID NO: 43 - protein, 16 aa, Corona virus SARS-CoV-2
MVMFTPLVPFWITIAY

SEQ ID NO: 44 - protein, 15 aa, Corona virus SARS-CoV-2
FYWFFSNYLKRRVVF

SEQ ID NO: 45 - protein, 14 aa, Corona virus SARS-CoV-2
LPSLATVAYFNMVY

SEQ ID NO: 46 - protein, 10 aa, Corona virus SARS-CoV-2
LILMTARTVY

SEQ ID NO: 47 - protein, 12 aa, Corona virus SARS-CoV-2
TACTDDNALAYY

SEQ ID NO: 48 - protein, 10 aa, Corona virus SARS-CoV-2
YTMADLVYAL

SEQ ID NO: 49 - protein, 14 aa, Corona virus SARS-CoV-2
KLFDRYFKYWDQTY

SEQ ID NO: 50 - protein, 10 aa, Corona virus SARS-CoV-2
ARLYYDSMSY

SEQ ID NO: 51 - protein, 19 aa, Corona virus SARS-CoV-2
VDTDFVNEFYAYLRKHFSM

SEQ ID NO: 52 - protein, 13 aa, Corona virus SARS-CoV-2
FPLCANGQVFGLY

SEQ ID NO: 53 - protein, 14 aa, Corona virus SARS-CoV-2
IPLMYKGLPWNVVR

SEQ ID NO: 54 - protein, 10 aa, Corona virus SARS-CoV-2
YVMHANYIFW

SEQ ID NO: 55 - protein, 37 aa, Corona virus SARS-CoV-2
SEETGTLIVNSVLLFLAFVVFLLVTLAILTALRLCAY

SEQ ID NO: 56 - protein, 21 aa, Corona virus SARS-CoV-2
VSLVKPSFYVYSRVKNLNSSR

SEQ ID NO: 57 - protein, 13 aa, Corona virus SARS-CoV-2
FMRIFTIGTVTLK

SEQ ID NO: 58 - protein, 36 aa, Corona virus SARS-CoV-2
TIPIQASLPFGWLIVGVALLAVFQSASKIITLKKRW

SEQ ID NO: 59 - protein, 13 aa, Corona virus SARS-CoV-2
HFVCNLLLLFVTV

SEQ ID NO: 60 - protein, 40 aa, Corona virus SARS-CoV-2
LLVAAGLEAPFLYLYALVYFLQSINFVRIIMRLWLCWKCR

SEQ ID NO: 61 - protein, 12 aa, Corona virus SARS-CoV-2
YFLCWHTNCYDY

SEQ ID NO: 62 - protein, 15 aa, Corona virus SARS-CoV-2
TTSPISEHDYQIGGY

SEQ ID NO: 63 - protein, 26 aa, Corona virus SARS-CoV-2
YYQLYSTQLSTDTGVEHVTFFIYNKI

SEQ ID NO: 64 - protein, 32 aa, Corona virus SARS-CoV-2
FHLVDFQVTIAEILLIIMRTFKVSIWNLDYII

SEQ ID NO: 65 - protein, 20 aa, Corona virus SARS-CoV-2
MKIILFLALITLATCELYHY

SEQ ID NO: 66 - protein, 60 aa, Corona virus SARS-CoV-2
LLKEPCSSGTYEGNSPFHPLADNKFALTCFSTQFAFACPDGVKHVYQLRARSVSPKLFIR

SEQ ID NO: 67 - protein, 28 aa, Corona virus SARS-CoV-2
EVQELYSPIFLIVAAIVFITLCFTLKRK

SEQ ID NO: 68 - protein, 34 aa, Corona virus SARS-CoV-2
MIELSLIDFYLCFLAFLLFLVLIMLIIFWFSLEL

SEQ ID NO: 69 - protein, 16 aa, Corona virus SARS-CoV-2
MKFLVFLGIITTVAAF

SEQ ID NO: 70 - protein, 31 aa, Corona virus SARS-CoV-2
YVVDDPCPIHFYSKWYIRVGARKSAPLIELC

SEQ ID NO: 71 - protein, 48 aa, Corona virus SARS-CoV-2
IQYIDIGNYTVSCLPFTINCQEPKLGSLVVRCSFYEDFLEYHDVRVVL

SEQ ID NO: 72 - protein, 48 aa, Corona virus SARS-CoV-2
IQYIDIGNYTVSCSPFTINCQEPKLGSLVVRCSFYEDFLEYHDVRVVL

SEQ ID NO: 73 - protein, 20 aa, Corona virus SARS-CoV-2
MGYINVFAFPFTIYSLLLCR

SEQ ID NO: 74 - protein, 22 aa, Corona virus SARS-CoV-2
VNSVLLFLAFVVFLLVTLAILT

SEQ ID NO: 75 - protein, 16 aa, Corona virus SARS-CoV-2
IKLIFLWLLWPVTLAC

SEQ ID NO: 76 - protein, 18 aa, Corona virus SARS-CoV-2
VGLMWLSYFIASFRLFAR

SEQ ID NO: 77 - protein, 19 aa, Corona virus SARS-CoV-2
SRTLSYYKLGASQRVAGDS

SEQ ID NO: 78 - protein, 21 aa, Corona virus SARS-CoV-2
SKTQSLLIVNNATNVVIKVCE

SEQ ID NO: 79 - protein, 17 aa, Corona virus SARS-CoV-2
REFVFKNIDGYFKIYSK

SEQ ID NO: 80 - protein, 38 aa, Corona virus SARS-CoV-2
GINITRFQTLLALHRSYLTPGDSSSGWTAGAAAYYVGY

SEQ ID NO: 81 - protein, 19 aa, Corona virus SARS-CoV-2
QPTESIVRFPNITNLCPFG

SEQ ID NO: 82 - protein, 17 aa, Corona virus SARS-CoV-2
ADYSVLYNSASFSTFKC

SEQ ID NO: 83 - protein, 19 aa, Corona virus SARS-CoV-2
YADSFVIRGDEVRQIAPGQ

SEQ ID NO: 84 - protein, 19 aa, Corona virus SARS-CoV-2
VVVLSFELLHAPATVCGPK

SEQ ID NO: 85 - protein, 19 aa, Corona virus SARS-CoV-2
IAIPTNFTISVTTEILPVS

SEQ ID NO: 86 - protein, 18 aa, Corona virus SARS-CoV-2
ECSNLLLQYGSFCTQLNR

SEQ ID NO: 87 - protein, 16 aa, Corona virus SARS-CoV-2
QQLIRAAEIRASANLA

SEQ ID NO: 88 - protein, 16 aa, Corona virus SARS-CoV-2
REGVFVSNGTHWFVTQ

SEQ ID NO: 89 - protein, 24 aa, Corona virus SARS-CoV-2
PWYIWLGFIAGLIAIVMVTIMLCC

SEQ ID NO: 90 - protein, 19 aa, Corona virus SARS-CoV-2
DDQIGYYRRATRRIRGGDG

SEQ ID NO: 91 - protein, 21 aa, Corona virus SARS-CoV-2
GTRNPANNAAIVLQLPQGTTL

SEQ ID NO: 92 - protein, 19 aa, Corona virus SARS-CoV-2
WPQIAQFAPSASAFFGMSR

SEQ ID NO: 93 - protein, 19 aa, Corona virus SARS-CoV-2
TPSGTWLTYTGAIKLDDKD

SEQ ID NO: 94 - protein, 17 aa, Corona virus SARS-CoV-2
SDFVRATATIPIQASLP

SEQ ID NO: 95 - protein, 19 aa, Corona virus SARS-CoV-2
APFLYLYALVYFLQSINFV

SEQ ID NO: 96 - protein, 23 aa, Corona virus SARS-CoV-2
DTGVEHVTFFIYNKIVDEPEEHV

SEQ ID NO: 97 - protein, 17 aa, Corona virus SARS-CoV-2
AIILASFSASTSAFVET

SEQ ID NO: 98 - protein, 16 aa, Corona virus SARS-CoV-2
YAFASEAARVVRSIFS

SEQ ID NO: 99 - protein, 19 aa, Corona virus SARS-CoV-2
TLEETKFLTENLLLYIDIN

SEQ ID NO: 100 - protein, 19 aa, Corona virus SARS-CoV-2
VQQESPFVMMSAPPAQYEL

SEQ ID NO: 101 - protein, 19 aa, Corona virus SARS-CoV-2
LLQLCTFTRSTNSRIKASM

SEQ ID NO: 102 - protein, 17 aa, Corona virus SARS-CoV-2
SKLINIIIWFLLLSVCL

SEQ ID NO: 103 - protein, 50 aa, Corona virus SARS-CoV-2
EWFLAYILFTRFFYVLGLAAIMQLFFSYFAVHFISNSWLMWLIINLVQMA

SEQ ID NO: 104 - protein, 18 aa, Corona virus SARS-CoV-2
VRMYIFFASFYYVWKSYV

SEQ ID NO: 105 - protein, 26 aa, Corona virus SARS-CoV-2
WLKQLIKVTLVFLFVAAIFYLITPVH

SEQ ID NO: 106 - protein, 18 aa, Corona virus SARS-CoV-2
FSAVGNICYTPSKLIEYT

SEQ ID NO: 107 - protein, 19 aa, Corona virus SARS-CoV-2
DISASIVAGGIVAIVVTCL

SEQ ID NO: 108 - protein, 19 aa, Corona virus SARS-CoV-2
FTPLVPFWITIAYIICIST

SEQ ID NO: 109 - protein, 19 aa, Corona virus SARS-CoV-2
VQSTQWSLFFFLYENAFLP

SEQ ID NO: 110 - protein, 18 aa, Corona virus SARS-CoV-2
QAIASEFSSLPSYAAFAT

SEQ ID NO: 111 - protein, 17 aa, Corona virus SARS-CoV-2
TQMNLKYAISAKNRART

SEQ ID NO: 112 - protein, 19 aa, Corona virus SARS-CoV-2
RAMPNMLRIMASLVLARKH

SEQ ID NO: 113 - protein, 18 aa, Corona virus SARS-CoV-2
FVNEFYAYLRKHFSMMIL

SEQ ID NO: 114 - protein, 16 aa, Corona virus SARS-CoV-2
PEVKILNNLGVDIAAN

SEQ ID NO: 115 - protein, 19 aa, Corona virus SARS-CoV-2
SQMEIDFLELAMDEFIERY

SEQ ID NO: 116 - protein, 16 aa, Corona virus SARS-CoV-2
FAWWTAFVTNVNASSS

SEQ ID NO: 117 - protein, 18 aa, Corona virus SARS-CoV-2
HANYIFWRNTNPIQLSSY

SEQ ID NO: 118 - protein, 26 aa, Corona virus SARS-CoV-2
EILLIIMRTFKVSIWNLDYIINLIIK

SEQ ID NO: 119 - protein, 18 aa, Corona virus SARS-CoV-2
MKIILFLALITLATCELY

SEQ ID NO: 120 - protein, 31 aa, Corona virus SARS-CoV-2
LSLIDFYLCFLAFLLFLVLIMLIIFWFSLEL

SEQ ID NO: 121 - protein, 19 aa, Corona virus SARS-CoV-2
CTQHQPYVVDDPCPIHFYS

SEQ ID NO: 122 - protein, 19 aa, Corona virus SARS-CoV-2
YINVFAFPFTIYSLLLCRM

SEQ ID NO: 123 - protein, 842 aa, Artificial sequence
  Vaccine construct
GIGDPVTCLKSGAICHPVFCPRRYKQIGTCGLPGTKCCKKPEAAAKGTITVEELKKLLEQWNLVIGFLFLTWICLLQFAYANRNRFLYIIKLIFLWLLWPVTLACFVLAAVGGGGSRINWITGGIAIAMACLVGLMWLSYFIASFRLFARTRSMWSFNGGGGSKEITVATSRTLSYYKLGASQRVAGDSGFAAYSRYRIGNYKLGGGGSTTRTQLPPAYTNSFTRGVYYPDKVFRSSVGGGGSSTQDLFLPFFSNVTWFGGGGSVLPFNDGVYFASTEKSNIIRGWIFGGGGSGVYYHKNNKSWMESEFRVYSSANNCTFEYVSQPFLGGGGSKQGNFKNLREFVFKNIDGYFKIYSKHTPIGGGGSEPLVDLPIGINITRFQTLLAGGGGSTPGDSSSGWTAGAAAYYVGYLQPRTFLLKGGGGSSETKCTLKSFTVEKGIYQTSNFRGGGGSFPNITNLCPFGEVFNATRFASVGGGGSRISNCVADYSVLYNSASFSTFKCYGGGGSNVYADSFVIRGDEVRGGGGSSKVGGNYNYLYRLFRKSNLKPFERGGGGSNTSNQVAVLYQDVNCTEVGGGGSRVYSTGSNVFGGGGSSPRRARSVASQSIIAYGGGGSFTISVTTEILPVSMTKTSVDCTMYGGGGSNTQEVFAQVKQIYKTPPIKGGGGSKRSFIEDLLFNKVTLAGGGGSPLLTDEMIAQYGGGGSLQIPFAMQMAYRFNGIGGGGSFPQSAPHGVVFGGGGSFVSNGTHWFVTQRGGGGSYEQYIKWPWYIWLGFIAGLIAIVEAAAKGIINTLQKYYCRVRGGRCAVLSCLPKEEQIGKCSTRGRKCCRRKKHHHHHH

SEQ ID NO: 124 - protein, 675 aa, Artificial sequence
  Vaccine construct
GIGDPVTCLKSGAICHPVFCPRRYKQIGTCGLPGTKCCKKPEAAAKNQRNAPRITFGGPSGGGGSRSKQRRPQGLPNNTASWFTALTQHGKGGGGSNSSPDDQIGYYRRATRRIRGGDGKMKDLSPRWYFYYLGGGGSLPYGANKDGIIWGGGGSKSAAEASKKPRQKRTATKAYNVTQAFGGGGSQELIRQGTDYGGGGSIAQFAPSASAFFGMSRIGMEVTPSGTWLTYGGGGSLLNKHIDAYKTFPPTEPKKGGGGSDEWSMATYYLFGGGGSETISLAGSYKGGGGSTQVVDMSMTYGGGGSAVMYMGTLSYEQFGGGGSLVAEWFLAYILFTRFFYVGGGGSRMYIFFASFYYVWKSYGGGGSAYVNTFSSTFNVPMEKGGGGSCLAYYFMRFRRAFGGGGSMVMFTPLVPFWITIAYGGGGSFYWFFSNYLKRRVVFGGGGSLPSLATVAYFNMVYGGGGSLILMTARTVYGGGGSTACTDDNALAYYGGGGSYTMADLVYALGGGGSKLFDRYFKYWDQTYGGGGSARLYYDSMSYGGGGSVDTDFVNEFYAYLRKHFSMGGGGSFPLCANGQVFGLYGGGGSIPLMYKGLPWNVVRGGGGSYVMHANYIFWEAAAKGIINTLQKYYCRVRGGRCAVLSCLPKEEQIGKCSTRGRKCCRRKKHHHHHH

SEQ ID NO: 125 - protein, 689 aa, Artificial sequence
  Vaccine construct
GIGDPVTCLKSGAICHPVFCPRRYKQIGTCGLPGTKCCKKPEAAAKSEETGTLIVNSVLLFLAFVVFLLVTLAILTALRLCAYGGGGSVSLVKPSFYVYSRVKNLNSSRGGGGSFMRIFTIGTVTLKGGGGSTIPIQASLPFGWLIVGVALLAVFQSASKIITLKKRWGGGGSHFVCNLLLLFVTVGGGGSLLVAAGLEAPFLYLYALVYFLQSINFVRIIMRLWLCWKCRGGGGSYFLCWHTNCYDYGGGGSTTSPISEHDYQIGGYGGGGSYYQLYSTQLSTDTGVEHVTFFIYNKIGGGGSFHLVDFQVTIAEILLIIMRTFKVSIWNLDYIIGGGGSMKIILFLALITLATCELYHYGGGGSLLKEPCSSGTYEGNSPFHPLADNKFALTCFSTQFAFACPDGVKHVYQLRARSVSPKLFIRGGGGSEVQELYSPIFLIVAAIVFITLCFTLKRKGGGGSMIELSLIDFYLCFLAFLLFLVLIMLIIFWFSLELGGGGSMKFLVFLGIITTVAAFGGGGSYVVDDPCPIHFYSKWYIRVGARKSAPLIELCGGGGSIQYIDIGNYTVSCLPFTINCQEPKLGSLVVRCSFYEDFLEYHDVRVVLGGGGSMGYINVFAFPFTIYSLLLCREAAAKGIINTLQKYYCRVRGGRCAVLSCLPKEEQIGKCSTRGRKCCRRKKHHHHHH

SEQ ID NO: 126 - protein, 644 aa, artificial sequence
  Vaccine construct
GIGDPVTCLKSGAICHPVFCPRRYKQIGTCGLPGTKCCKKPEAAAKVNSVLLFLAFVVFLLVTLAILTGGGGSIKLIFLWLLWPVTLACGGGGSVGLMWLSYFIASFRLFARGGGGSSRTLSYYKLGASQRVAGDSGGGGSSKTQSLLIVNNATNVVIKVCEGGGGSREFVFKNIDGYFKIYSKGGGGSGINITRFQTLLALHRSYLTPGDSSSGWTAGAAAYYVGYGGGGSQPTESIVRFPNITNLCPFGGGGGSADYSVLYNSASFSTFKCGGGGSYADSFVIRGDEVRQIAPGQGGGGSVVVLSFELLHAPATVCGPKGGGGSIAIPTNFTISVTTEILPVSGGGGSQQLIRAAEIRASANLAGGGGSREGVFVSNGTHWFVTQGGGGSPWYIWLGFIAGLIAIVMVTIMLCCGGGGSDDQIGYYRRATRRIRGGDGGGGGSGTRNPANNAAIVLQLPQGTTLGGGGSWPQIAQFAPSASAFFGMSRGGGGSTPSGTWLTYTGAIKLDDKDGGGGSSDFVRATATIPIQASLPGGGGSAPFLYLYALVYFLQSINFVGGGGSDTGVEHVTFFIYNKIVDEPEEHVEAAAKGIINTLQKYYCRVRGGRCAVLSCLPKEEQIGKCSTRGRKCCRRKKHHHHHH

SEQ ID NO: 127 - protein, 757 aa, artificial sequence
  Vaccine construct
GIGDPVTCLKSGAICHPVFCPRRYKQIGTCGLPGTKCCKKPEAAAKAIILASFSASTSAFVETGGGGSYAFASEAARVVRSIFSGGGGSTLEETKFLTENLLLYIDINGGGGSVQQESPFVMMSAPPAQYELGGGGSLLQLCTFTRSTNSRIKASMGGGGSSKLINIIIWFLLLSVCLGGGGSEWFLAYILFTRFFYVLGLAAIMQLFFSYFAVHFISNSWLMWLIINLVQMAGGGGSVRMYIFFASFYYVWKSYVGGGGSWLKQLIKVTLVFLFVAAIFYLITPVHGGGGSFSAVGNICYTPSKLIEYTGGGGSDISASIVAGGIVAIVVTCLGGGGSFTPLVPFWITIAYIICISTGGGGSVQSTQWSLFFFLYENAFLPGGGGSQAIASEFSSLPSYAAFATGGGGSTQMNLKYAISAKNRARTGGGGSRAMPNMLRIMASLVLARKHGGGGSFVNEFYAYLRKHFSMMILGGGGSPEVKILNNLGVDIAANGGGGSSQMEIDFLELAMDEFIERYGGGGSFAWWTAFVTNVNASSSGGGGSHANYIFWRNTNPIQLSSYGGGGSEILLIIMRTFKVSIWNLDYIINLIIKGGGGSMKIILFLALITLATCELYGGGGSLSLIDFYLCFLAFLLFLVLIMLIIFWFSLELGGGGSCTQHQPYVVDDPCPIHFYSGGGGSYINVFAFPFTIYSLLLCRMEAAAKGIINTLQKYYCRVRGGRCAVLSCLPKEEQIGKCSTRGRKCCRRKKHHHHHH

SEQ ID NO: 128 - protein, 41 aa, Corona virus SARS-CoV-2
GIGDPVTCLKSGAICHPVFCPRRYKQIGTCGLPGTKCCKKP

SEQ ID NO: 129 - protein, 45 aa, Corona virus SARS-CoV-2
GIINTLQKYYCRVRGGRCAVLSCLPKEEQIGKCSTRGRKCCRRKK

SEQ ID NO: 130 - protein, 5 aa, artificial sequence
  linker sequence
GGGGS

SEQ ID NO: 131 - protein, 5 aa, artificial sequence
  linker sequence
EAAAK

SEQ ID NO: 132 - protein, 6 aa, artificial sequence
  His-tag
HHHHHH

SEQ ID NO: 133 - protein, 13 aa, Corona virus SARS-CoV-2
STEEEKDDIKNGK

SEQ ID NO: 134 - protein, 13 aa, Corona virus SARS-CoV-2
KNQENNLTLLPIK

SEQ ID NO: 135 - protein, 13 aa, Corona virus SARS-CoV-2
NLTLLPIKSTEEE

SEQ ID NO: 136 - protein, 13 aa, Corona virus SARS-CoV-2
IKSTEEEKDDIKN

SEQ ID NO: 137 - protein, 13 aa, Corona virus SARS-CoV-2
TLLPIKSTEEEKD

SEQ ID NO: 138 - protein, 9 aa, Corona virus SARS-CoV-2
EEEKDDIKN

SEQ ID NO: 139 - protein, 24 aa, Corona virus SARS-CoV-2
KNQENNLTLLPIKSTEEEKDDIKN

SEQ ID NO: 140 - protein, 13 aa, Corona virus SARS-CoV-2
KKEIDNDKENIKT

SEQ ID NO: 141 - protein, 13 aa, Corona virus SARS-CoV-2
DKENIKTRYTPRG

SEQ ID NO: 142 - protein, 13 aa, Corona virus SARS-CoV-2
KTRYTPRGALVRP

SEQ ID NO: 143 - protein, 13 aa, Corona virus SARS-CoV-2
ENIKTRYTPRGAL

SEQ ID NO: 144 - protein, 13 aa, Corona virus SARS-CoV-2
GALVRPWDDGKKN

SEQ ID NO: 145 - protein, 31 aa, Corona virus SARS-CoV-2
KKEIDNDKENIKTRYTPRGALVRPWDDGKKN

SEQ ID NO: 146 - protein, 10 aa, Corona virus SARS-CoV-2
SQRVAGDSGF

SEQ ID NO: 147 - protein, 11 aa, Corona virus SARS-CoV-2
KEITVATSRTL

SEQ ID NO: 148 - protein, 9 aa, Corona virus SARS-CoV-2
FAAYSRYRI

SEQ ID NO: 149 - protein, 10 aa, Corona virus SARS-CoV-2
AYSRYRIGNY

SEQ ID NO: 150 - protein, 9 aa, Corona virus SARS-CoV-2
YSRYRIGNY

SEQ ID NO: 151 - protein, 10 aa, Corona virus SARS-CoV-2
YSRYRIGNYK

SEQ ID NO: 152 - protein, 8 aa, Corona virus SARS-CoV-2
RYRIGNYK

SEQ ID NO: 153 - protein, 9 aa, Corona virus SARS-CoV-2
RYRIGNYKL

SEQ ID NO: 154 - protein, 10 aa, Corona virus SARS-CoV-2
DSGFAAYSRY

SEQ ID NO: 155 - protein, 9 aa, Corona virus SARS-CoV-2
SGFAAYSRY

SEQ ID NO: 156 - protein, 9 aa, Corona virus SARS-CoV-2
GFAAYSRYR

SEQ ID NO: 157 - protein, 10 aa, Corona virus SARS-CoV-2
SGFAAYSRYR

SEQ ID NO: 158 - protein, 10 aa, Corona virus SARS-CoV-2
SYYKLGASQR

SEQ ID NO: 159 - protein, 9 aa, Corona virus SARS-CoV-2
YYKLGASQR

SEQ ID NO: 160 - protein, 10 aa, Corona virus SARS-CoV-2
TVATSRTLSY

SEQ ID NO: 161 - protein, 9 aa, Corona virus SARS-CoV-2
VATSRTLSY

SEQ ID NO: 162 - protein, 10 aa, Corona virus SARS-CoV-2
VATSRTLSYY

SEQ ID NO: 163 - protein, 9 aa, Corona virus SARS-CoV-2
TSRTLSYYK

SEQ ID NO: 164 - protein, 9 aa, Corona virus SARS-CoV-2
ATSRTLSYY

SEQ ID NO: 165 - protein, 10 aa, Corona virus SARS-CoV-2
ATSRTLSYYK

SEQ ID NO: 166 - protein, 11 aa, Corona virus SARS-CoV-2
VATSRTLSYYK

SEQ ID NO: 167 - protein, 10 aa, Corona virus SARS-CoV-2
ILTALRLCAY

SEQ ID NO: 168 - protein, 9 aa, Corona virus SARS-CoV-2
LTALRLCAY

SEQ ID NO: 169 - protein, 9 aa, Corona virus SARS-CoV-2
TLAILTALR

SEQ ID NO: 170 - protein, 10 aa, Corona virus SARS-CoV-2
VTLAILTALR

SEQ ID NO: 171 - protein, 9 aa, Corona virus SARS-CoV-2
VLLFLAFVV

SEQ ID NO: 172 - protein, 10 aa, Corona virus SARS-CoV-2
SVLLFLAFVV

SEQ ID NO: 173 - protein, 10 aa, Corona virus SARS-CoV-2
NSVLLFLAFV

SEQ ID NO: 174 - protein, 9 aa, Corona virus SARS-CoV-2
SVLLFLAFV

SEQ ID NO: 175 - protein, 10 aa, Corona virus SARS-CoV-2
VLLFLAFVVF

SEQ ID NO: 176 - protein, 9 aa, Corona virus SARS-CoV-2
LLFLAFVVF

SEQ ID NO: 177 - protein, 10 aa, Corona virus SARS-CoV-2
LLFLAFVVFL

SEQ ID NO: 178 - protein, 9 aa, Corona virus SARS-CoV-2
VFLLVTLAI

SEQ ID NO: 179 - protein, 9 aa, Corona virus SARS-CoV-2
SEETGTLIV

SEQ ID NO: 180 - protein, 9 aa, Corona virus SARS-CoV-2
LIVNSVLLF

SEQ ID NO: 181 - protein, 9 aa, Corona virus SARS-CoV-2
LAFVVFLLV

SEQ ID NO: 182 - protein, 9 aa, Corona virus SARS-CoV-2
FVVFLLVTL

SEQ ID NO: 183 - protein, 9 aa, Corona virus SARS-CoV-2
FLLVTLAIL

SEQ ID NO: 184 - protein, 9 aa, Corona virus SARS-CoV-2
FLAFVVFLL

SEQ ID NO: 185 - protein, 10 aa, Corona virus SARS-CoV-2
FLAFVVFLLV

SEQ ID NO: 186 - protein, 11 aa, Corona virus SARS-CoV-2
LFLAFVVFLLV

SEQ ID NO: 187 - protein, 8 aa, Corona virus SARS-CoV-2
KMKDLSPR

SEQ ID NO: 188 - protein, 10 aa, Corona virus SARS-CoV-2
KMKDLSPRWY

SEQ ID NO: 189 - protein, 10 aa, Corona virus SARS-CoV-2
DLSPRWYFYY

SEQ ID NO: 190 - protein, 9 aa, Corona virus SARS-CoV-2
LSPRWYFYY

SEQ ID NO: 191 - protein, 10 aa, Corona virus SARS-CoV-2
KDLSPRWYFY

SEQ ID NO: 192 - protein, 11 aa, Corona virus SARS-CoV-2
MKDLSPRWYFY

SEQ ID NO: 193 - protein, 9 aa, Corona virus SARS-CoV-2
YYRRATRRI

SEQ ID NO: 194 - protein, 10 aa, Corona virus SARS-CoV-2
YYRRATRRIR

SEQ ID NO: 195 - protein, 11 aa, Corona virus SARS-CoV-2
NSSPDDQIGYY

SEQ ID NO: 196 - protein, 9 aa, Corona virus SARS-CoV-2
SPDDQIGYY

SEQ ID NO: 197 - protein, 10 aa, Corona virus SARS-CoV-2
SSPDDQIGYY

SEQ ID NO: 198 - protein, 9 aa, Corona virus SARS-CoV-2
SPRWYFYYL

SEQ ID NO: 199 - protein, 9 aa, Corona virus SARS-CoV-2
IGYYRRATR

SEQ ID NO: 200 - protein, 9 aa, Corona virus SARS-CoV-2
GYYRRATRR